(19) United States
(12) Patent Application Publication
(10) Pub. No.: US 2013/0085303 A1
(43) Pub. Date: Apr. 4, 2013

(54) Processes for Producing Acrylic Acids and Acrylates

(71) Applicant: Celanese International Corporation, Irving, TX (US)

(72) Inventors: Craig J. Peterson, Houston, TX (US); Josefina T. Chapman, Houston, TX (US)

(73) Assignee: CELANESE INTERNATIONAL CORPORATION, Irving, TX (US)

(21) Appl. No.: 13/632,813

(22) Filed: Oct. 1, 2012

Related U.S. Application Data
(63) Continuation-in-part of application No. 13/251,623, filed on Oct. 3, 2011.

(51) Int. Cl. C07C 51/42

(52) U.S. Cl. 562/600

(57) ABSTRACT

In one embodiment, the invention is to a process for producing an acrylate product. The process comprises the step of providing a crude product stream comprising the acrylate product, an alkylenating agent, light ends, and non-condensable gases. The process further comprises the step of separating the crude product stream to form a cooled vapor stream and at least one condensed crude product stream without the addition of heat. The process further comprise the step of separating at least a portion of the condensed crude product stream to form an alkylenating agent stream comprising at least 1 wt. % alkylenating agent and the intermediate product stream comprises acrylate product.

### CROSS REFERENCE TO RELATED APPLICATION

This application is a continuation-in-part to U.S. application Ser. No. 13/251,623, filed on Oct. 3, 2011, the entirety of which is incorporated herein by reference.

### FIELD OF THE INVENTION

The present invention relates generally to the production of acrylic acid. More specifically, the present invention relates to the production of crude acrylic acid via the condensation of acetic acid and formaldehyde and the subsequent purification thereof.

### BACKGROUND OF THE INVENTION

α,β-unsaturated acids, particularly acrylic acid and methacrylic acid, and the ester derivatives thereof are useful organic compounds in the chemical industry. These acids and esters are known to readily polymerize or co-polymerize to form homopolymers or copolymers. Often the polymerized acids are useful in applications such as superabsorbents, dispersants, flocculants, and thickeners. The polymerized ester derivatives are used in coatings (including latex paints), textiles, adhesives, plastics, fibers, and synthetic resins.

Because acrylic acid and its esters have long been valued commercially, many methods of production have been developed. One exemplary acrylic acid ester production process utilizes: (1) the reaction of acetylene with water and carbon monoxide; and/or (2) the reaction of an alcohol and carbon monoxide, in the presence of an acid, e.g., hydrochloric acid, and nickel tetracarbonyl, to yield a crude product comprising the acrylate ester as well as hydrogen and nickel chloride. Another conventional process involves the reaction of ketene (often obtained by the pyrolysis of acetone or acetic acid) with formaldehyde, which yields a crude product comprising acrylic acid and either water (when acetic acid is used as a pyrolysis reactant) or methane (when acetone is used as a pyrolysis reactant). These processes have become obsolete for economic, environmental, or other reasons.

More recent acrylic acid production processes have relied on the gas phase oxidation of propylene, via acrolein, to form acrylic acid. The reaction can be carried out in single- or two-step processes but the latter is favored because of higher yields. The oxidation of propylene produces acrolein, acrylic acid, acetaldehyde and carbon oxides. Acrylic acid from the primary oxidation can be recovered while the acrolein is fed to a second step to yield the crude acrylic acid product, which comprises acrylic acid, water, small amounts of acetic acid, as well as impurities such as furfural, acrolein, and propionic acid. Purification of the crude product may be carried out by azeotropic distillation. Although this process may show some improvement over earlier processes, this process suffers from production and/or separation inefficiencies. In addition, this oxidation reaction is highly exothermic and, as such, creates an explosion risk. As a result, more expensive reactor design and metallurgy are required. Also, the cost of propylene is often prohibitive.

The aldol condensation reaction of formaldehyde and acetic acid and/or carboxylic acid esters has been disclosed in literature. This reaction forms acrylic acid and is often conducted over a catalyst. For example, condensation catalysts consisting of mixed oxides of vanadium and phosphorus were investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987). The acetic acid conversions in these reactions, however, may leave room for improvement. Although this reaction is disclosed, there has been little if any disclosure relating to separation schemes that may be employed to effectively provide purified acrylic acid from the aldol condensation crude product.

U.S. Pat. App. 2012/0071688 teaches a process for preparing acrylic acid from methanol and acetic acid. In a first reaction zone, methanol is partially oxidized to formaldehyde in a heterogeneously catalyzed gas phase reaction to obtain a first product gas mixture. Excess amount of acetic acid is added to the first product gas mixture to obtain a second product, which comprises unreacted acetic acid and formaldehyde. The formaldehyde and acetic acid is aldo-condensed to form a product mixture including acrylic acid and unreacted acetic acid under heterogeneous catalysis. The unreacted acetic acid in the product mixture is removed and recycled into the production of the second product.

Thus, the need exists for processes for producing purified acrylic acid and, in particular, for separation schemes that effectively purify the unique aldol condensation crude products to form the purified acrylic acid.

The references mentioned above are hereby incorporated by reference.

### BRIEF DESCRIPTION OF DRAWINGS

The invention is described in detail below with reference to the appended drawings, wherein like numerals designate similar parts.

FIG. 1 is a process flowsheet showing an acrylic acid reaction/separation system in accordance with an embodiment of the present invention.

FIG. 2 is a schematic diagram of light ends and non-condensable gases removal in accordance with an embodiment of the present invention.

FIG. 3 is a schematic diagram of light ends and non-condensable gases removal in accordance with an embodiment of the present invention.

FIG. 4 is a schematic diagram of light ends and non-condensable gases removal in accordance with an embodiment of the present invention.

FIG. 5 is a schematic diagram of an acrylic acid reaction/separation system in accordance with one embodiment of the present invention.

### SUMMARY OF THE INVENTION

In one embodiment, the invention is to a process for producing an acrylate product, such as acrylic acid, methacrylic acid, and/or the salts and esters thereof. Preferably, the inventive process yields an acrylic acid product. The process comprises the step of providing a crude product stream comprising the acrylate product, an alkylenating agent, light ends, and non-condensable gases. In one embodiment, the inventive process further comprises the step of separating the crude product stream to form a cooled vapor stream and a condensed crude product stream. Preferably, the separating is performed without the addition of heat. In one embodiment, the inventive process further comprises the step of separating at least a portion of the condensed crude product stream to form an alkylenating agent stream and an intermediate product stream. Preferably, the alkylenating stream comprises at least 1 wt. % alkylenating agent and the intermediate acrylic product stream comprises acrylate product.

In one embodiment, the process comprises the step of cooling the crude product stream using a first heat exchanger to form a first vapor stream and a first liquid stream. The process may further comprise the step of adding inhibitors to the first liquid stream. The process further comprises the step of reducing the temperature of the crude product stream with one or more cooled derivative streams.

In one embodiment, the process comprises the step of separating the crude product stream in a rectifying column to form a vapor stream and a residue stream. In one embodiment, the process comprises the step of separating the crude product stream in a quench column to form a vapor stream and a residue stream.

### DETAILED DESCRIPTION OF THE INVENTION

### Introduction

Production of unsaturated carboxylic acids such as acrylic acid and methacrylic acid and the ester derivatives thereof via most conventional processes have been limited by economic and environmental constraints. In the interest of finding a new reaction path, the aldol condensation reaction of acetic acid and an alkylenating agent, e.g., formaldehyde, has been investigated. This reaction may yield a unique crude product that comprises, inter alia, a higher amount of (residual) formaldehyde, which is generally known to add unpredictability and problems to separation schemes.

The unique crude product may comprise light ends and non-condensable gases. These light ends and non-condensable gases require removal from the system for the recovery of the desired acrylic acid product. The inventors have found that the removal of these light ends and non-condensable gases earlier in the purification system surprisingly and unexpectedly improves separation efficiencies and yields higher purity acrylic acid products. Without being bound by theory, it is believed that additional by-products may be formed when some of the light ends and/or non-condensable gases contact with methyl acrylate (which may also be considered a light ends). These additional by-products may complicate the purification of the crude acrylate product stream and lead to separation inefficiencies. For example, methanol may react with acetic acid to form methyl acetate and methyl acetate may react with acrylic acid to form methyl acrylate. Therefore, by removing light ends such as methanol and methyl acetate, build-up of these compounds and the formation of byproducts may be prevented. In addition, methyl acrylate is a reactive monomer, which may cause fouling problems if it reaches sufficient concentrations.

Furthermore, the removal of the light ends and non-condensable gases from the crude acrylate product stream advantageously reduces the size of the crude acrylate product stream and, as such, may beneficially reduce the burden on the downstream separation columns used to purify the crude acrylate product. As a result, smaller separation columns that require less energy to operate may be used. Thus, the removal of light ends and non-condensable gases from the crude product stream beneficially reduces the overall cost of the production of acrylic acid.

Although the aldol condensation reaction of acetic acid and formaldehyde is known, there has been little if any disclosure relating to separation schemes that may be employed to effectively purify the unique crude product that is produced. Other conventional reactions, e.g., propylene oxidation or ketene/formaldehyde, do not yield crude products that comprise higher amounts of formaldehyde. The primary reactions and the side reactions in propylene oxidation do not create formaldehyde. In the reaction of ketene and formaldehyde, a two-step reaction is employed and the formaldehyde is confined to the first stage. Also, the ketene is highly reactive and converts substantially all of the reactant formaldehyde. As a result of these features, very little, if any, formaldehyde remains in the crude product exiting the reaction zone. Because no formaldehyde is present in crude products formed by these conventional reactions, the separation schemes associated therewith have not addressed the problems and unpredictability that accompany crude products that have higher formaldehyde content.

In one embodiment, the present invention relates to a process for producing acrylic acid, methacrylic acid, and/or the salts and esters thereof. As used herein, acrylic acid, methacrylic acid, and/or the salts and esters thereof, collectively or individually, may be referred to as “acrylate products.” The use of the terms acrylic acid, methacrylic acid, or the salts and esters thereof, individually, does not exclude the other acrylate products, and the use of the term acrylate product does not require the presence of acrylic acid, methacrylic acid, and the salts and esters thereof.

The inventive process, in one embodiment, includes the step of providing a crude product stream comprising the acrylic acid and/or other acrylate products. The crude product stream of the present invention, unlike most conventional acrylic acid-containing crude products, further comprises a significant portion of at least one alkylenating agent. Preferably, the at least one alkylenating agent is formaldehyde. For example, the crude product stream may comprise at least 0.5 wt. % alkylenating agent(s), e.g., at least 1 wt. %, at least 5 wt. %, at least 7 wt. %, at least 10 wt. %, or at least 25 wt. %. In terms of ranges, the crude product stream may comprise from 0.5 wt. % to 50 wt. % alkylenating agent(s), e.g., from 1 wt. % to 45 wt. %, from 1 wt. % to 25 wt. %, from 1 wt. % to 10 wt. %, or from 5 wt. % to 10 wt. %. In terms of upper limits, the crude product stream may comprise less than 50 wt. % alkylenating agent(s), e.g., less than 45 wt. %, less than 25 wt. %, or less than 10 wt. %.

In one embodiment, the crude product stream further comprises one or more light ends and/or non-condensable gases. For example the crude product stream may comprise non-condensable gases, such as oxygen, nitrogen, carbon monoxide, carbon dioxide, and hydrogen, and/or light ends, such as methanol, methyl acetate, methyl acrylate, acetaldehyde, and acetone. In one embodiment, the crude product stream may comprise at least 20 wt. % light ends and/or non-condensable gases, e.g., at least 30 wt. % or at least 50 wt. %. In terms of ranges, the crude product stream may comprise from 20 wt. % to 90 wt. % light ends and/or non-condensable gases, e.g., from 30 wt. % to 80 wt. %, or from 50 wt. % to 70 wt. %. In terms of upper limits, the crude product stream may comprise at most 90 wt. % light ends and/or non-condensable gases, e.g., at most 80 wt. %, or at most 70 wt. %.

In one embodiment, the crude product stream of the present invention further comprises water. For example, the crude product stream may comprise less than 60 wt. % water, e.g., less than 50 wt. %, less than 40 wt. %, or less than 30 wt. %. In terms of ranges, the crude product stream may comprise from 1 wt. % to 60 wt. % water, e.g., from 5 wt. % to 50 wt. %, from 10 wt. % to 40 wt. %, or from 15 wt. % to 40 wt. %. In terms of lower limits, the crude product stream may comprise at least 1 wt. % water, e.g., at least 5 wt. %, at least 10 wt. %, or at least 15 wt. %.

In one embodiment, the crude product stream of the present invention comprises very little, if any, of the impurities found in most conventional acrylic acid crude product streams. For example, the crude product stream of the present invention may comprise less than 1000 wppm of such impurities (either as individual components or collectively), e.g., less than 500 wppm, less than 100 wppm, less than 50 wppm, or less than 10 wppm. Exemplary impurities include acetylene, ketene, beta-propiolactone, higher alcohols, e.g., C₂₊, C₃₊, or C₄₊, and combinations thereof. Importantly, the crude product stream of the present invention comprises very little, if any, furfural and/or acrolein. In one embodiment, the crude product stream comprises substantially no furfural and/or acrolein, e.g., no furfural and/or acrolein. In one embodiment, the crude product stream comprises less than less than 500 wppm acrolein, e.g., less than 100 wppm, less than 50 wppm, or less than 10 wppm. In one embodiment, the crude product stream comprises less than less than 500 wppm furfural, e.g., less than 100 wppm, less than 50 wppm, or less than 10 wppm. Furfural and acrolein are known to act as detrimental chain terminators in acrylic acid polymerization reactions. Also, furfural and/or acrolein are known to have adverse effects on the color of purified product and/or to subsequent polymerized products.

In addition to the acrylic acid and the alkylenating agent, the crude product stream may further comprise acetic acid, and propionic acid.

Exemplary compositional data for the crude product stream are shown in Table 1. Components other than those listed in Table 1 may also be present in the crude product stream.

TABLE 1
CRUDE ACRYLATE PRODUCT STREAM COMPOSITIONS

| | Conc. | Conc. | Conc. | Conc. |
|---|---|---|---|---|
| Component | (wt. %) | (wt. %) | (wt. %) | (wt. %) |
| Acrylic Acid | 1 to 75 | 1 to 50 | 5 to 50 | 10 to 40 |
| Alkylenating Agent(s) | 0.5 to 50 | 1 to 45 | 1 to 25 | 1 to 10 |
| Acetic Acid | 1 to 90 | 1 to 70 | 5 to 50 | 10 to 50 |
| Water | 1 to 60 | 5 to 50 | 10 to 40 | 15 to 40 |
| Propionic Acid | 0.01 to 10 | 0.1 to 10 | 0.1 to 5 | 0.1 to 1 |
| Oxygen | 0.01 to 10 | 0.1 to 10 | 0.1 to 5 | 0.1 to 1 |
| Nitrogen | 0.1 to 20 | 0.1 to 10 | 0.5 to 5 | 0.5 to 4 |
| Carbon Monoxide | 0.01 to 10 | 0.1 to 10 | 0.1 to 5 | 0.5 to 3 |
| Carbon Dioxide | 0.01 to 10 | 0.1 to 10 | 0.1 to 5 | 0.5 to 3 |
| Other Light Ends | 0.01 to 10 | 0.1 to 10 | 0.1 to 5 | 0.5 to 3 |

The unique crude product stream of the present invention may be separated in a separation zone to form a final product, e.g., a final acrylic acid product. In one embodiment, the inventive process reduces the size of the crude product stream by removing light ends and non-condensable gases from the crude product stream. As noted above, by removing the light ends and/or non-condensable gases from the crude acrylate product stream upstream of the additional components of the separation zone, the energy burden on the additional components is significantly reduced, as compared to a similar separation zone in which the light ends and/or non-condensable gases are not first removed. In one embodiment, the inventive process comprises the step of separating at least a portion of the crude acrylate product stream to form at least one cooled vapor stream and at least one condensed crude product stream. Preferably, the cooled vapor stream(s) comprise light ends and non-condensable gases and the condensed crude product stream(s) comprises acrylate product. Preferably, the separation of the crude product stream is performed without the application of heat.

The separation scheme used to separate the light ends and/or non-condensable gases from the crude acrylate product may vary widely. In one embodiment, one or more separation unit is used to separate the light ends and/or non-condensable gases from the crude acrylate product. In an embodiment, the one or more separation unit may comprise one or more heat exchangers and or flashers or knock-out pot. In one embodiment, the heat exchangers may be used to cool the crude product stream. The cooled crude product stream may be sent to a knock-out pot or flasher. In one embodiment, the temperature of the crude acrylate product stream is from 200° C. to 600° C., e.g., from 250° C. to 500° C. or from 340° C. to 425° C.

As a result of the cooling process using the first heat exchanger, a cooled crude product stream may be separated into a first vapor stream and a first liquid stream. As a result of the cooling, the first liquid stream has a temperature lower than the temperature of the crude product stream. For example, the temperature of the first liquid stream may range from 10° C. to 120° C., e.g., from 15° C. to 80° C. or from 30° C. to 50° C. In one embodiment, the first liquid stream may be separated and a portion of which may be sent to a second heat exchanger. The second heat exchanger cools the first liquid stream to yield a cooled first liquid pump around stream. For example, the temperature of the cooled first liquid (pump around) stream may range from 1° C. to 50° C., e.g., from 5° C. to 40° C. or from 10° C. to 30° C. In preferred embodiments, the cooled first liquid (pump around) stream may be recycled and used as a cooling stream to cool the crude product stream prior to the crude product steam entering into the first heat exchanger. For example, the inventive process may comprise the step of combining at least a portion of the cooled first liquid stream with the crude product stream, thus cooling the crude product stream. In another embodiment, the pump around stream, which contains inhibitor), is sprayed into the heat exchanger to prevent the formation of polymer and increases operability. The use of the cooled first liquid stream to cool the crude product stream may beneficially lower the energy requirements of the first heat exchanger.

As stated above, the crude product stream may be separated into a first liquid stream and a first vapor stream. The temperature of the first vapor stream may be from 10° C. to 120° C., e.g., from 15° C. to 80° C. or from 30° C. to 50° C. The first vapor stream comprises mostly light ends and non-condensable gases. For example, the first vapor stream comprises from 20 wt. % to 99 wt. % light ends and non-condensable gases, e.g., from 60 wt. % to 95 wt. %, or from 88 wt. % to 93 wt. %. The first vapor stream may also comprise condensable components such as acrylate products, alkylenating agent, acrylic acid, water, and other components. For example, the first vapor stream may comprise from 0.001 wt. % to 8 wt. % acrylate products, e.g., from 0.1 wt. % to 5 wt. %, or from 0.5 wt. % to 2 wt. %. It is beneficial to recover additional amount of acrylate product. Therefore, the first vapor stream may be sent to a second separation unit to further condense the vapor stream to recover additional condensable components.

In an embodiment, the first liquid stream comprises less than 1 wt. % light ends compounds and non-condensable gases, e.g., less than 0.1 wt. % or less than 0.001 wt. %. In an embodiment, the first liquid stream may comprise greater than 55 wt. % acrylate products, e.g., greater than 70 wt. %, or greater than 85 wt. %. As such, the first liquid stream is the condensed crude product stream, which is further separated to yield an acrylate product.

In one embodiment, the first vapor stream is cooled in a second separation unit, which comprises at least one heat exchanger and at least one flasher or knock-out pot. For example, the temperature of the cooled first vapor stream is from 1° C. to 50° C., e.g., from 5° C. to 40° C. or from 10° C. to 30° C. The cooled first vapor stream may be separated into a second vapor stream and a second liquid stream. The second liquid stream may be further treated to form a condensed product stream. The temperature of the second vapor stream may be from 1° C. to 50° C., e.g., from 5° C. to 40° C. or from 10° C. to 30° C.

The second vapor stream comprises mostly light ends and non-condensable gases. For example, the second vapor stream comprises from 80 wt. % to 99.999 wt. % light ends and non-condensable gases, e.g., from 90 wt. % to 99.5 wt. %, or from 95 wt. % to 99 wt. %. In an embodiment, the second vapor stream comprises less condensable gases by weight percentage than the first vapor stream. For example, the second vapor stream comprises less than 9 wt. % condensable products, e.g., less than 5 wt. % or less than 3 wt. %. In an embodiment, the condensable components may include acrylate products, alkylenating agent, acrylic acid and/or water. In an embodiment, the second vapor stream comprises less than 5 wt. % acrylics, e.g., less than 1 wt. % or less than 0.1 wt. %.

The temperature of the second liquid stream may be from 1° C. to 50° C., e.g., from 5° C. to 40° C. or from 10° C. to 30° C. In one embodiment, the second liquid stream may be separated. A portion of the second liquid stream may form a second liquid pump around stream, which may be used to cool the first vapor stream prior to entry into the second separation unit.

In an embodiment, the second liquid stream comprises less than 1 wt. % light ends compounds and non-condensable gases, e.g., less than 0.1 wt. % or less than 0.05 wt. %. In an embodiment, the second liquid stream may comprise from 1 wt. % to 45 wt. % acrylate products, e.g., from 5 wt. % to 35 wt. %, or from 10 wt. % to 25 wt. %. In one embodiment, the second liquid pump around stream may be combined with the first liquid stream to form the condensed crude product stream. In one embodiment, the condensed crude product stream comprises less than 1 wt. % light ends compounds and non-condensable gases, less than 0.5 wt. %, or less than 0.1 wt. %. In one embodiment, the condensed crude product stream comprises at least 0.5 wt. % alkylenating agent, e.g., at least 5 wt. % or at least 20 wt. %.

In some embodiments, polymerization inhibitors may be added to one or more streams to prevent the acrylate product, e.g., acrylic acid, from polymerizing in the heat exchanger. For example, a polymerization inhibitor feed may be introduced to a portion of the first liquid stream which may serve as a cooling stream for the crude product stream. The amount of polymerization inhibitors used typically depends on the content of the acrylic acid. In an embodiment, 0.01 wt. % to 5 wt. % polymerization inhibitor may be added to the first liquid stream, e.g., 0.01 wt. % to 1 wt. %, or 0.01 wt. % to 0.05 wt. %.

Useful polymerization inhibitors here are, for example, alkylphenols, e.g. o-, m- or p-cresol (methylphenol), 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, 4-tert-butyl-2,6-dimethylphenol, or 2,2′-methylenebis-(6-tert-butyl-4-methylphenol), hydroxyphenols, e.g. hydroquinone, 2-methylhydroquinone, 2,5-di-tert-butylhydroquinone, pyrocatechol (1,2-dihydroxybenzene) or benzoquinone, aminophenols, e.g. para-aminophenol, nitrosophenols, e.g. para-nitrosophenol, alkoxyphenols, e.g. 2-methoxyphenol (guaiacol, pyrocatechol monomethyl ether), 2-ethoxyphenol, 2-isopropoxyphenol, 4-methoxyphenol (hydroquinone monomethyl ether), mono- or di-tert-butyl-4-methoxyphenol, tocopherols and also 2,3-dihydro-2,2-dimethyl-7-hydroxybenzofuran (2,2-dimethyl-7-hydroxycoumaran), N-oxyls such as 4-hydroxy-2,2,6,6-tetramethylpiperidine N-oxyl, 4-oxo-2,2,6,6-tetramethylpiperidine N-oxyl, 4-acetoxy-2,2,6,6-tetramethylpiperidine N-oxyl, 2,2,6,6-tetramethylpiperidine N-oxyl, 4,4′,4″-tris(2,2,6,6-tetramethylpiperidine N-oxyl) phosphite or 3-oxo-2,2,5,5-tetramethylpyrrolidine N-oxyl, aromatic amines or phenylenediamines, e.g. N,N-diphenylamine, N-nitrosodiphenylamine, N,N′-dialkylparaphenylenediamine in which the alkyl radicals may be the same or different and each independently contain from 1 to 4 carbon atoms and may be straight-chain or branched, hydroxylamines, e.g. N,N-diethylhydroxylamine, phosphorus compounds, e.g. triphenyl-phosphine, triphenyl phosphite, hypophosphorous acid or triethyl phosphite, sulfur compounds, e.g. diphenyl sulfide or phenothiazine, optionally in combination with metal salts, for example the chlorides, dithiocarbamates, sulfates, salicylates or acetates of copper, manganese, cerium, nickel or chromium. It will be appreciated that mixtures of stabilizers can also be used.

In one embodiment, a rectifying column may be used to remove light ends and non-condensable gases from the crude acrylate product. In one embodiment, the crude acrylate product stream is fed directly to the rectifying column. In one embodiment, the crude acrylate product stream is in vapor form and is fed directly to the rectifying column without being condensed. It is postulated that the feeding of the crude vapor stream to the rectification column effectively separates light ends and non-condensable gases from the condensable components of the crude product stream. Furthermore, the feeding of the crude vapor product into the rectifying column eliminates the need for a reboiler, e.g., the separation may be conducted without the addition of heat. Therefore, the potential for acrylic polymerization is advantageously reduced.

In an embodiment, the crude product vapor stream is introduced at the bottom half of the rectifying column, e.g., bottom third, or bottom quarter. In a preferred embodiment, one or more polymerization inhibitors may be added to the rectifying column. In an embodiment, the one or more inhibitors may be added at the top half of the rectifying column, e.g., top third, or top quarter. The use of polymerization inhibitor is to limit the undesired polymer formation because polymer formation may undesirably increases the pressure drop over the rectification column. Furthermore, the formation of polymers reduces the amount of product formed and reduces the separation efficiency of the column. In other embodiments, the inhibitors may be added to the crude product stream. In one embodiment, a pump around stream may be used on the rectifying column.

In an embodiment, the crude acrylate product stream is separated into a vapor stream and a residue stream, e.g., a condensed crude acrylate product stream. For example, the vapor stream may comprise light components, such as nitrogen, oxygen, carbon dioxides and carbon monoxides, and may exit overhead. The residue stream may comprise formaldehyde, acetic acid, acrylic acid and propionic acid. In one embodiment, the residue stream comprises less than 10 wt. % light ends compounds and non-condensable gases, e.g., less than 5 wt. % or less than 1 wt. %. In an embodiment, the residue stream may comprise from 1 wt. % to 60 wt. % acrylate products, e.g., from 15 wt. % to 50 wt. %, or from 20 wt. % to 40 wt. %.

In one embodiment, the temperature of the residue exiting the rectification column ranges from 50° C. to 150° C., e.g., from 75° C. to 130° C. or from 90° C. to 115° C. The temperature of the vapor stream exiting the rectification column preferably ranges from 50° C. to 150° C., e.g., from 75° C. to 130° C. or from 90° C. to 115° C. The pressure at which the rectification column is operated may range from 10 kPa to 110 kPa, e.g., from 50 kPa to 110 kPa or from 90 kPa to 110 kPa. In preferred embodiments, to prevent undesirable polymerization of acrylic acid, the pressure at which the rectification column is operated is kept at a low level e.g., less than 110 kPa, less than 108 kPa, or less than 105 kPa. In terms of lower limits, the rectification column may be operated at a pressures of at least 10 kPa, e.g., at least 50 kPa or at least 90 kPa.

In one embodiment, a quench column may be used to remove light ends and non-condensable gases from the crude acrylate product. In one embodiment, the crude acrylate product stream is fed directly to the quench column. In one embodiment, the crude acrylate product stream is in vapor form and is fed directly to the quench column without being condensed. One or more solvent is used as a quenching agent.

In one embodiment, the crude acrylate product vapor stream is introduced at the bottom of the quenching column, e.g., bottom third, or bottom quarter. In one embodiment, a quenching solvent is introduced at the top of the quenching column, e.g., top third, or top quarter. The temperature of the quench solvent entering the quench column preferably ranges from 0° C. to 70° C., e.g., from 20° C. to 60° C. or from 30° C. to 50° C. In one embodiment, one or more polymerization inhibitor may be added to the quench column. In one embodiment, the one or more polymerization inhibitor may be added with the quenching solvent. The use of polymerization inhibitor is to limit the undesired polymer formation in the residue.

In one embodiment, the crude acrylate product stream is separated into a vapor stream and a residue stream, e.g., condensed crude acrylate product stream. In an embodiment, a side stream is withdrawn from the bottom of the quenching column, e.g., bottom third, or bottom quarter. The side stream is returned to the quench column at a higher location, e.g., top half, top third, or top quarter. As such, the side stream is also known as a pump around stream.

In one embodiment, the pump around stream exits from the quench column at a location higher than the crude acrylate product. In one embodiment, the pump around stream enters into the quench column at a location lower than the quench solvent feed. In one embodiment, the pump around stream is passes through a heat exchanger before reentering into the quench column. Therefore, the heat exchanger reduces the temperature of the pump around stream when it reenters the quench column. In one embodiment, the temperature of the pump around stream exiting the quench column ranges from 30° C. to 100° C., e.g., from 40° C. to 90° C. or from 45° C. to 80° C. The temperature of the pump around stream reentering the quench column preferably ranges from 0° C. to 70° C., e.g., from 20° C. to 60° C. or from 30° C. to 50° C. In one embodiment, the polymerization inhibitor may be added to the pump around stream. In one embodiment, more than one pump around stream may be used.

In one embodiment, the vapor stream may comprise light components, such as nitrogen, oxygen, carbon dioxides and carbon monoxides, and may exit overhead. The residue, e.g., condensed crude product, stream may comprise formaldehyde, acetic acid, acrylic acid and propionic acid. In one embodiment, the residue stream comprises less than 1 wt. % light ends compounds and non-condensable gases, e.g., less than 0.1 wt. % or less than 0.05 wt. %. In an embodiment, the residue stream may comprise from 1 wt. % to 60 wt. % acrylate products, e.g., from 15 wt. % to 50 wt. %, or from 20 wt. % to 40 wt. %.

In one embodiment, the temperature of the residue exiting the quench column ranges from 50° C. to 150° C., e.g., from 75° C. to 130° C. or from 90° C. to 115° C. The temperature of the vapor stream exiting the quench column preferably ranges from 0° C. to 70° C., e.g., from 20° C. to 60° C. or from 30° C. to 50° C. The pressure at which the quench column is operated may range from 10 kPa to 110 kPa, e.g., from 50 kPa to 110 kPa or from 90 kPa to 110 kPa. In preferred embodiments, to prevent undesirable polymerization of acrylic acid, the pressure at which the quench column is operated is kept at a low level e.g., less than 110 kPa, less than 108 kPa, or less than 105 kPa. In one embodiment, the quench column is operated at atmospheric pressure. In terms of lower limits, the quench column may be operated at a pressures of at least 10 kPa, e.g., at least 50 kPa or at least 90 kPa.

In one embodiment, the inventive process comprises the step of separating at least a portion of the condensed crude product stream to form an alkylenating agent stream and an intermediate product stream. This separating step may be referred to as an “alkylenating agent split.” In one embodiment, the alkylenating agent stream comprises significant amounts of alkylenating agent(s). For example, the alkylenating agent stream may comprise at least 1 wt. % alkylenating agent(s), e.g., at least 5 wt. %, at least 10 wt. %, at least 15 wt. %, or at least 25 wt. %. In terms of ranges, the alkylenating stream may comprise from 1 wt. % to 75 wt. % alkylenating agent(s), e.g., from 3 to 50 wt. %, from 3 wt. % to 25 wt. %, or from 10 wt. % to 20 wt. %. In terms of upper limits, the alkylenating stream may comprise less than 75 wt. % alkylenating agent(s), e.g. less than 50 wt. % or less than 40 wt. %. In preferred embodiments, the alkylenating agent is formaldehyde.

As noted above, the presence of alkylenating agent in the crude product stream adds unpredictability and problems to separation schemes. Without being bound by theory, it is believed that formaldehyde reacts in many side reactions with water to form by-products. The following side reactions are exemplary.

CH₂O+H₂O→HOCH₂OH

HO(CH₂O)ᵢ₋₁H+HOCH₂OH→HO(CH₂O)ᵢH+H₂O for i>1

Without being bound by theory, it is believed that, in some embodiments, as a result of these reactions, the alkylenating agent, e.g., formaldehyde, acts as a “light” component at higher temperatures and as a “heavy” component at lower temperatures. The reaction(s) are exothermic. Accordingly, the equilibrium constant increases as temperature decreases and decreases as temperature increases. At lower temperatures, the larger equilibrium constant favors methylene glycol and oligomer production and formaldehyde becomes limited, and, as such, behaves as a heavy component. At higher temperatures, the smaller equilibrium constant favors formaldehyde production and methylene glycol becomes limited. As such, formaldehyde behaves as a light component. In view of these difficulties, as well as others, the separation of streams that comprise water and formaldehyde cannot be expected to behave as a typical two-component system. These features contribute to the unpredictability and difficulty of the separation of the unique crude product stream of the present invention.

The present invention, surprisingly and unexpectedly, achieves effective separation of alkylenating agent(s) from the inventive crude product stream to yield a purified product comprising acrylate product and very low amounts of other impurities.

In one embodiment, the alkylenating split is performed such that a lower amount of acetic acid is present in the resulting alkylenating stream. Preferably, the alkylenating agent stream comprises little or no acetic acid. As an example, the alkylenating agent stream, in some embodiments, comprises less than 50 wt. % acetic acid, e.g., less than 45 wt. %, less than 25 wt. %, less than 10 wt. %, less than 5 wt. %, less than 3 wt. %, or less than 1 wt. %. Surprisingly and unexpectedly, the present invention provides for the lower amounts of acetic acid in the alkylenating agent stream, which, beneficially reduces or eliminates the need for further treatment of the alkylenating agent stream to remove acetic acid. In some embodiments, the alkylenating agent stream may be treated to remove water therefrom, e.g., to purge water.

In some embodiments, the alkylenating agent split is performed in at least one column, e.g., at least two columns or at least three columns. Preferably, the alkylenating agent is performed in a two column system. In other embodiments, the alkylenating agent split is performed via contact with an extraction agent. In other embodiments, the alkylenating agent split is performed via precipitation methods, e.g., crystallization, and/or azeotropic distillation. Of course, other suitable separation methods may be employed either alone or in combination with the methods mentioned herein.

The intermediate product stream comprises acrylate products. In one embodiment, the intermediate product stream comprises a significant portion of acrylate products, e.g., acrylic acid. For example, the intermediate product stream may comprise at least 5 wt. % acrylate products, e.g., at least 25 wt. %, at least 40 wt. %, at least 50 wt. %, or at least 60 wt. %. In terms of ranges, the intermediate product stream may comprise from 5 wt. % to 99 wt. % acrylate products, e.g. from 10 wt. % to 90 wt. %, from 25 wt. % to 75 wt. %, or from 35 wt. % to 65 wt. %. The intermediate product stream, in one embodiment, comprises little if any alkylenating agent. For example, the intermediate product stream may comprise less than 1 wt. % alkylenating agent, e.g., less than 0.1 wt. % alkylenating agent, less than 0.05 wt. %, or less than 0.01 wt. %. In addition to the acrylate products, the intermediate product stream optionally comprises acetic acid, water, propionic acid and other components.

In some cases, the intermediate acrylate product stream comprises higher amounts of alkylenating agent. For example, in one embodiment, the intermediate acrylate product stream comprises from 1 wt. % to 50 wt. % alkylenating agent, e.g., from 1 wt. % to 10 wt. % or from 5 wt. % to 50 wt. %. In terms of limits, the intermediate acrylate product stream may comprise at least 1 wt. % alkylenating agent, e.g., at least 5 wt. % or at least 10 wt. %.

In one embodiment, the crude product stream is optionally treated, e.g. separated, prior to the separation of alkylenating agent therefrom. In such cases, the treatment(s) occur before the alkylenating agent split is performed. In other embodiments, at least a portion of the intermediate acrylate product stream may be further treated after the alkylenating agent split. As one example, the crude product stream may be treated to remove light ends therefrom. This treatment may occur either before or after the alkylenating agent split, preferably before the alkylenating agent split. In some of these cases, the further treatment of the intermediate acrylate product stream may result in derivative streams that may be considered to be additional purified acrylate product streams. In other embodiments, the further treatment of the intermediate acrylate product stream results in at least one finished acrylate product stream.

In one embodiment, the inventive process operates at a high process efficiency. For example, the process efficiency may be at least 10%, e.g., at least 20% or at least 35%. In one embodiment, the process efficiency is calculated based on the flows of reactants into the reaction zone. The process efficiency may be calculated by the following formula.

Process Efficiency=2N_{HAcA}/[N_{HOAc}+N_{HCHO}+N_{H2O}]

where:

N_{HAcA} is the molar production rate of acrylate products; and

N_{HOAc}, N_{HCHO}, and N_{H2O} are the molar feed rates of acetic acid, formaldehyde, and water.

### Production of Acrylate Products

Any suitable reaction and/or separation scheme may be employed to form the crude product stream as long as the reaction provides the crude product stream components that are discussed above. For example, in some embodiments, the acrylate product stream is formed by contacting an alkanoic acid, e.g., acetic acid, or an ester thereof with an alkylenating agent, e.g., a methylenating agent, for example formaldehyde, under conditions effective to form the crude acrylate product stream. Preferably, the contacting is performed over a suitable catalyst. The crude product stream may be the reaction product of the alkanoic acid-alkylenating agent reaction. In a preferred embodiment, the crude product stream is the reaction product of the aldol condensation reaction of acetic acid and formaldehyde, which is conducted over a catalyst comprising vanadium and titanium. In one embodiment, the crude product stream is the product of a reaction in wherein methanol and acetic acid are combined to generate formaldehyde in situ. The aldol condensation then follows. In one embodiment, a methanol-formaldehyde solution is reacted with acetic acid to form the crude product stream.

The alkanoic acid, or an ester of the alkanoic acid, may be of the formula R′—CH₂—COOR, where R and R′ are each, independently, hydrogen or a saturated or unsaturated alkyl or aryl group. As an example, R and R′ may be a lower alkyl group containing for example 1-4 carbon atoms. In one embodiment, an alkanoic acid anhydride may be used as the source of the alkanoic acid. In one embodiment, the reaction is conducted in the presence of an alcohol, preferably the alcohol that corresponds to the desired ester, e.g., methanol. In addition to reactions used in the production of acrylic acid, the inventive catalyst, in other embodiments, may be employed to catalyze other reactions.

The alkanoic acid, e.g., acetic acid, may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. As petroleum and natural gas prices fluctuate, becoming either more or less expensive, methods for producing acetic acid and intermediates such as methanol and carbon monoxide from alternate carbon sources have drawn increasing interest. In particular, when petroleum is relatively expensive compared to natural gas, it may become advantageous to produce acetic acid from synthesis gas (“syngas”) that is derived from any available carbon source. U.S. Pat. No. 6,232,352, which is hereby incorporated by reference, for example, teaches a method of retrofitting a methanol plant for the manufacture of acetic acid. By retrofitting a methanol plant, the large capital costs associated with carbon monoxide generation for a new acetic acid plant are significantly reduced or largely eliminated. All or part of the syngas is diverted from the methanol synthesis loop and supplied to a separator unit to recover carbon monoxide and hydrogen, which are then used to produce acetic acid.

In some embodiments, at least some of the raw materials for the above-described aldol condensation process may be derived partially or entirely from syngas. For example, the acetic acid may be formed from methanol and carbon monoxide, both of which may be derived from syngas. For example, the methanol may be formed by steam reforming syngas, and the carbon monoxide may be separated from syngas. In other embodiments, the methanol may be formed in a carbon monoxide unit, e.g., as described in EP2076480; EP1923380; EP2072490; EP1914219; EP1904426; EP2072487; EO2072492; EP2072486; EP2060553; EP1741692; EP1907344; EP2060555; EP2186787; EP2072488; and U.S. Pat. No. 7,842,844, which are hereby incorporated by reference. Of course, this listing of methanol sources is merely exemplary and is not meant to be limiting. In addition, the above-identified methanol sources, inter alia, may be used to form the formaldehyde, e.g., in situ, which, in turn may be reacted with the acetic acid to form the acrylic acid. The syngas, in turn, may be derived from variety of carbon sources. The carbon source, for example, may be selected from the group consisting of natural gas, oil, petroleum, coal, biomass, and combinations thereof.

Methanol carbonylation processes suitable for production of acetic acid are described in U.S. Pat. Nos. 7,208,624, 7,115,772, 7,005,541, 6,657,078, 6,627,770, 6,143,930, 5,599,976, 5,144,068, 5,026,908, 5,001,259, and 4,994,608, all of which are hereby incorporated by reference.

U.S. Pat. No. RE 35,377, which is hereby incorporated by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syngas. The syngas is converted to methanol which may be carbonylated to acetic acid. U.S. Pat. No. 5,821,111, which discloses a process for converting waste biomass through gasification into syngas, as well as U.S. Pat. No. 6,685,754 are hereby incorporated by reference.

In one optional embodiment, the acetic acid that is utilized in the condensation reaction comprises acetic acid and may also comprise other carboxylic acids, e.g., propionic acid, esters, and anhydrides, as well as acetaldehyde and acetone. In one embodiment, the acetic acid fed to the condensation reaction comprises propionic acid. For example, the acetic acid fed to the reaction may comprise from 0.001 wt. % to 15 wt. % propionic acid, e.g., from 0.001 wt. % to 13 wt. %, from 0.125 wt. % to 12.5 wt. %, from 1.25 wt. % to 11.25 wt. %, or from 3.75 wt. % to 8.75 wt. %. Thus, the acetic acid feed stream may be a cruder acetic acid feed stream, e.g., a less-refined acetic acid feed stream.

As used herein, “alkylenating agent” means an aldehyde or precursor to an aldehyde suitable for reacting with the alkanoic acid, e.g., acetic acid, to form an unsaturated acid, e.g., acrylic acid, or an alkyl acrylate. In preferred embodiments, the alkylenating agent comprises a methylenating agent such as formaldehyde, which preferably is capable of adding a methylene group (═CH₂) to the organic acid. Other alkylenating agents may include, for example, acetaldehyde, propanal, butanal, aryl aldehydes, benzyl aldehydes, alcohols, and combinations thereof. This listing is not exclusive and is not meant to limit the scope of the invention. In one embodiment, an alcohol may serve as a source of the alkylenating agent. For example, the alcohol may be reacted in situ to form the alkylenating agent, e.g., the aldehyde.

The alkylenating agent, e.g., formaldehyde, may be derived from any suitable source. Exemplary sources may include, for example, aqueous formaldehyde solutions, anhydrous formaldehyde derived from a formaldehyde drying procedure, trioxane, diether of methylene glycol, and paraformaldehyde. In a preferred embodiment, the formaldehyde is produced via a methanol oxidation process, which reacts methanol and oxygen to yield the formaldehyde.

In other embodiments, the alkylenating agent is a compound that is a source of formaldehyde. Where forms of formaldehyde that are not as freely or weakly complexed are used, the formaldehyde will form in situ in the condensation reactor or in a separate reactor prior to the condensation reactor. Thus for example, trioxane may be decomposed over an inert material or in an empty tube at temperatures over 350° C. or over an acid catalyst at over 100° C. to form the formaldehyde.

In one embodiment, the alkylenating agent corresponds to Formula I.

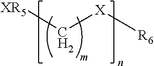

In this formula, R₅ and R₆ may be independently selected from C₁-C₁₂ hydrocarbons, preferably, C₁-C₁₂ alkyl, alkenyl or aryl, or hydrogen. Preferably, R₅ and R₆ are independently C₁-C₆ alkyl or hydrogen, with methyl and/or hydrogen being most preferred. X may be either oxygen or sulfur, preferably oxygen; and n is an integer from 1 to 10, preferably 1 to 3. In some embodiments, m is 1 or 2, preferably 1.

In one embodiment, the compound of formula I may be the product of an equilibrium reaction between formaldehyde and methanol in the presence of water. In such a case, the compound of formula I may be a suitable formaldehyde source. In one embodiment, the formaldehyde source includes any equilibrium composition. Examples of formaldehyde sources include but are not restricted to methylal (1,1dimethoxymethane); polyoxymethylenes —(CH₂—O)ᵢ— wherein i is from 1 to 100; formalin; and other equilibrium compositions such as a mixture of formaldehyde, methanol, and methyl propionate. In one embodiment, the source of formaldehyde is selected from the group consisting of 1,1 dimethoxymethane; higher formals of formaldehyde and methanol; and CH₃—O—(CH₂—O)ᵢ—CH₃ where i is 2.

The alkylenating agent may be used with or without an organic or inorganic solvent.

The term “formalin,” refers to a mixture of formaldehyde, methanol, and water. In one embodiment, formalin comprises from 25 wt. % to 65% formaldehyde; from 0.01 wt. % to 25 wt. % methanol; and from 25 wt. % to 70 wt. % water. In cases where a mixture of formaldehyde, methanol, and methyl propionate is used, the mixture comprises less than 10 wt. % water, e.g., less than 5 wt. % or less than 1 wt. %.

In some embodiments, the condensation reaction may achieve favorable conversion of acetic acid and favorable selectivity and productivity to acrylates. For purposes of the present invention, the term “conversion” refers to the amount of acetic acid in the feed that is converted to a compound other than acetic acid. Conversion is expressed as a percentage based on acetic acid in the feed. The conversion of acetic acid may be at least 10%, e.g., at least 20%, at least 40%, or at least 50%.

Selectivity, as it refers to the formation of acrylate product, is expressed as the ratio of the amount of carbon in the desired product(s) and the amount of carbon in the total products. This ratio may be multiplied by 100 to arrive at the selectivity. Preferably, the catalyst selectivity to acrylate products, e.g., acrylic acid and methyl acrylate, is at least 40 mol %, e.g., at least 50 mol %, at least 60 mol %, or at least 70 mol %. In some embodiments, the selectivity to acrylic acid is at least 30 mol %, e.g., at least 40 mol %, or at least 50 mol %; and/or the selectivity to methyl acrylate is at least 10 mol %, e.g., at least 15 mol %, or at least 20 mol %.

The terms “productivity” or “space time yield” as used herein, refers to the grams of a specified product, e.g., acrylate products, formed per hour during the condensation based on the liters of catalyst used. A productivity of at least 20 grams of acrylate product per liter catalyst per hour, e.g., at least 40 grams of acrylates per liter catalyst per hour or at least 100 grams of acrylates per liter catalyst per hour, is preferred. In terms of ranges, the productivity preferably is from 20 to 500 grams of acrylates per liter catalyst per hour, e.g., from 20 to 200 per kilogram catalyst per hour or from 40 to 140 per kilogram catalyst per hour.

In one embodiment, the inventive process yields at least 1,800 kg/hr of finished acrylic acid, e.g., at least 3,500 kg/hr, at least 18,000 kg/hr, or at least 37,000 kg/hr.

Preferred embodiments of the inventive process demonstrate a low selectivity to undesirable products, such as carbon monoxide and carbon dioxide. The selectivity to these undesirable products preferably is less than 29%, e.g., less than 25% or less than 15%. More preferably, these undesirable products are not detectable. Formation of alkanes, e.g., ethane, may be low, and ideally less than 2%, less than 1%, or less than 0.5% of the acetic acid passed over the catalyst is converted to alkanes, which have little value other than as fuel.

The alkanoic acid or ester thereof and alkylenating agent may be fed independently or after prior mixing to a reactor containing the catalyst. The reactor may be any suitable reactor or combination of reactors. Preferably, the reactor comprises a fixed bed reactor or a series of fixed bed reactors. In one embodiment, the reactor is a packed bed reactor or a series of packed bed reactors. In one embodiment, the reactor is a fixed bed reactor. Of course, other reactors such as a continuous stirred tank reactor or a fluidized bed reactor, may be employed.

In some embodiments, the alkanoic acid, e.g., acetic acid, and the alkylenating agent, e.g., formaldehyde, are fed to the reactor at a molar ratio of at least 0.10:1, e.g., at least 0.75:1 or at least 1:1. In terms of ranges the molar ratio of alkanoic acid to alkylenating agent may range from 0.10:1 to 10:1 or from 0.75:1 to 5:1. In some embodiments, the reaction of the alkanoic acid and the alkylenating agent is conducted with a stoichiometric excess of alkanoic acid. In these instances, acrylate selectivity may be improved. As an example the acrylate selectivity may be at least 10% higher than a selectivity achieved when the reaction is conducted with an excess of alkylenating agent, e.g., at least 20% higher or at least 30% higher. In other embodiments, the reaction of the alkanoic acid and the alkylenating agent is conducted with a stoichiometric excess of alkylenating agent.

The condensation reaction may be conducted at a temperature of at least 250° C., e.g., at least 300° C., or at least 350° C. In terms of ranges, the reaction temperature may range from 200° C. to 500° C., e.g., from 250° C. to 400° C., or from 250° C. to 350° C. Residence time in the reactor may range from 1 second to 200 seconds, e.g., from 1 second to 100 seconds. Reaction pressure is not particularly limited, and the reaction is typically performed near atmospheric pressure. In one embodiment, the reaction may be conducted at a pressure ranging from 0 kPa to 4,100 kPa, e.g., from 3 kPa to 345 kPa, or from 6 to 103 kPa. The acetic acid conversion, in some embodiments, may vary depending upon the reaction temperature.

In one embodiment, the reaction is conducted at a gas hourly space velocity (“GHSV”) greater than 600 hr⁻¹, e.g., greater than 1,000 hr⁻¹ or greater than 2,000 hr⁻¹. In one embodiment, the GHSV ranges from 600 hr⁻¹ to 10,000 hr⁻¹, e.g., from 1,000 hr⁻¹ to 8,000 hr⁻¹ or from 1,500 hr⁻¹ to 7,500 hr⁻¹. As one particular example, when GHSV is at least 2,000 hr⁻¹, the acrylate product STY may be at least 150 g/hr/liter.

Water may be present in the reactor in amounts up to 60 wt. %, by weight of the reaction mixture, e.g., up to 50 wt. % or up to 40 wt. %. Water, however, is preferably reduced due to its negative effect on process rates and separation costs.

In one embodiment, an inert or reactive gas is supplied to the reactant stream. Examples of inert gases include, but are not limited to, nitrogen, helium, argon, and methane. Examples of reactive gases or vapors include, but are not limited to, oxygen, carbon oxides, sulfur oxides, and alkyl halides. When reactive gases such as oxygen are added to the reactor, these gases, in some embodiments, may be added in stages throughout the catalyst bed at desired levels as well as feeding with the other feed components at the beginning of the reactors. The addition of these additional components may improve reaction efficiencies.

In one embodiment, the unreacted components such as the alkanoic acid and formaldehyde as well as the inert or reactive gases that remain are recycled to the reactor after sufficient separation from the desired product.

When the desired product is an unsaturated ester made by reacting an ester of an alkanoic acid ester with formaldehyde, the alcohol corresponding to the ester may also be fed to the reactor either with or separately to the other components. For example, when methyl acrylate is desired, methanol may be fed to the reactor. The alcohol, amongst other effects, reduces the quantity of acids leaving the reactor. It is not necessary that the alcohol is added at the beginning of the reactor and it may for instance be added in the middle or near the back, in order to effect the conversion of acids such as propionic acid, methacrylic acid to their respective esters without depressing catalyst activity. In one embodiment, the alcohol may be added downstream of the reactor.

### Catalyst Composition

The catalyst may be any suitable catalyst composition. As one example, condensation catalyst consisting of mixed oxides of vanadium and phosphorus have been investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987). Other examples include binary vanadium-titanium phosphates, vanadium-silica-phosphates, and alkali metal-promoted silicas, e.g., cesium- or potassium-promoted silicas.

In a preferred embodiment, the inventive process employs a catalyst composition comprising vanadium, titanium, and optionally at least one oxide additive. The oxide additive(s), if present, are preferably present in the active phase of the catalyst. In one embodiment, the oxide additive(s) are selected from the group consisting of silica, alumina, zirconia, and mixtures thereof or any other metal oxide other than metal oxides of titanium or vanadium. Preferably, the molar ratio of oxide additive to titanium in the active phase of the catalyst composition is greater than 0.05:1, e.g., greater than 0.1:1, greater than 0.5:1, or greater than 1:1. In terms of ranges, the molar ratio of oxide additive to titanium in the inventive catalyst may range from 0.05:1 to 20:1, e.g., from 0.1:1 to 10:1, or from 1:1 to 10:1. In these embodiments, the catalyst comprises titanium, vanadium, and one or more oxide additives and has relatively high molar ratios of oxide additive to titanium.

In other embodiments, the catalyst may further comprise other compounds or elements (metals and/or non-metals). For example, the catalyst may further comprise phosphorus and/or oxygen. In these cases, the catalyst may comprise from 15 wt. % to 45 wt. % phosphorus, e.g., from 20 wt. % to 35 wt. % or from 23 wt. % to 27 wt. %; and/or from 30 wt. % to 75 wt. % oxygen, e.g., from 35 wt. % to 65 wt. % or from 48 wt. % to 51 wt. %.

In some embodiments, the catalyst further comprises additional metals and/or oxide additives. These additional metals and/or oxide additives may function as promoters. If present, the additional metals and/or oxide additives may be selected from the group consisting of copper, molybdenum, tungsten, nickel, niobium, and combinations thereof. Other exemplary promoters that may be included in the catalyst of the invention include lithium, sodium, magnesium, aluminum, chromium, manganese, iron, cobalt, calcium, yttrium, ruthenium, silver, tin, barium, lanthanum, the rare earth metals, hafnium, tantalum, rhenium, thorium, bismuth, antimony, germanium, zirconium, uranium, cesium, zinc, and silicon and mixtures thereof. Other modifiers include boron, gallium, arsenic, sulfur, halides, Lewis acids such as BF₃, ZnBr₂, and SnCl₄. Exemplary processes for incorporating promoters into catalyst are described in U.S. Pat. No. 5,364,824, the entirety of which is incorporated herein by reference. In a preferred embodiment, the catalyst of the process of the present invention includes bismuth, tungsten, and mixtures thereof.

If the catalyst comprises additional metal(s) and/or metal oxides(s), the catalyst optionally may comprise additional metals and/or metal oxides in an amount from 0.001 wt. % to 30 wt. %, e.g., from 0.01 wt. % to 5 wt. % or from 0.1 wt. % to 5 wt. %. If present, the promoters may enable the catalyst to have a weight/weight space time yield of at least 25 grams of acrylic acid/gram catalyst-h, e.g., least 50 grams of acrylic acid/gram catalyst-h, or at least 100 grams of acrylic acid/gram catalyst-h.

In some embodiments, the catalyst is unsupported. In these cases, the catalyst may comprise a homogeneous mixture or a heterogeneous mixture as described above. In one embodiment, the homogeneous mixture is the product of an intimate mixture of vanadium and titanium oxides, hydroxides, and phosphates resulting from preparative methods such as controlled hydrolysis of metal alkoxides or metal complexes. In other embodiments, the heterogeneous mixture is the product of a physical mixture of the vanadium and titanium phosphates. These mixtures may include formulations prepared from phosphorylating a physical mixture of preformed hydrous metal oxides. In other cases, the mixture(s) may include a mixture of preformed vanadium pyrophosphate and titanium pyrophosphate powders.

In another embodiment, the catalyst is a supported catalyst comprising a catalyst support in addition to the vanadium, titanium, oxide additive, and optionally phosphorous and oxygen, in the amounts indicated above (wherein the molar ranges indicated are without regard to the moles of catalyst support, including any vanadium, titanium, oxide additive, phosphorous or oxygen contained in the catalyst support). The total weight of the support (or modified support), based on the total weight of the catalyst, preferably is from 75 wt. % to 99.9 wt. %, e.g., from 78 wt. % to 97 wt. % or from 80 wt. % to 95 wt. %. The support may vary widely. In one embodiment, the support material is selected from the group consisting of silica, alumina, zirconia, titania, aluminosilicates, zeolitic materials, mixed metal oxides (including but not limited to binary oxides such as SiO₂—Al₂O₃, SiO₂—TiO₂, SiO₂—ZnO, SiO₂—MgO, SiO₂—ZrO₂, Al₂O₃—MgO, Al₂O₃—TiO₂, Al₂O₃—ZnO, TiO₂—MgO, TiO₂—ZrO₂, TiO₂—ZnO, TiO₂—SnO₂) and mixtures thereof, with silica being one preferred support. In embodiments where the catalyst comprises a titania support, the titania support may comprise a major or minor amount of rutile and/or anatase titanium dioxide. Other suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, silicon carbide, sheet silicates or clay minerals such as montmorillonite, beidellite, saponite, pillared clays, other microporous and mesoporous materials, and mixtures thereof. Other supports may include, but are not limited to, iron oxide, magnesia, steatite, magnesium oxide, carbon, graphite, high surface area graphitized carbon, activated carbons, and mixtures thereof. These listings of supports are merely exemplary and are not meant to limit the scope of the present invention.

In some embodiments, a zeolitic support is employed. For example, the zeolitic support may be selected from the group consisting of montmorillonite, NH₄ ferrierite, H-mordenite-PVOx, vermiculite-1, H-ZSM5, NaY, H-SDUSY, Y zeolite with high SAR, activated bentonite, H-USY, MONT-2, HY, mordenite SAR 20, SAPO-34, Aluminosilicate (X), VUSY, Aluminosilicate (CaX), Re-Y, and mixtures thereof. H-SDUSY, VUSY, and H-USY are modified Y zeolites belonging to the faujasite family. In one embodiment, the support is a zeolite that does not contain any metal oxide modifier(s). In some embodiments, the catalyst composition comprises a zeolitic support and the active phase comprises a metal selected from the group consisting of vanadium, aluminum, nickel, molybdenum, cobalt, iron, tungsten, zinc, copper, titanium cesium bismuth, sodium, calcium, chromium, cadmium, zirconium, and mixtures thereof. In some of these embodiments, the active phase may also comprise hydrogen, oxygen, and/or phosphorus.

In other embodiments, in addition to the active phase and a support, the inventive catalyst may further comprise a support modifier. A modified support, in one embodiment, relates to a support that includes a support material and a support modifier, which, for example, may adjust the chemical or physical properties of the support material such as the acidity or basicity of the support material. In embodiments that use a modified support, the support modifier is present in an amount from 0.1 wt. % to 50 wt. %, e.g., from 0.2 wt. % to 25 wt. %, from 0.5 wt. % to 15 wt. %, or from 1 wt. % to 8 wt. %, based on the total weight of the catalyst composition.

In one embodiment, the support modifier is an acidic support modifier. In some embodiments, the catalyst support is modified with an acidic support modifier. The support modifier similarly may be an acidic modifier that has a low volatility or little volatility. The acidic modifiers may be selected from the group consisting of oxides of Group IVB metals, oxides of Group VB metals, oxides of Group VIB metals, iron oxides, aluminum oxides, and mixtures thereof. In one embodiment, the acidic modifier may be selected from the group consisting of WO₃, MoO₃, Fe₂O₃, Cr₂O₃, V₂O₅, MnO₂, CuO, Co₂O₃, Bi₂O₃, TiO₂, ZrO₂, Nb₂O₅, Ta₂O₅, Al₂O₃, B₂O₃, P₂O₅, and Sb₂O₃.

In another embodiment, the support modifier is a basic support modifier. The presence of chemical species such as alkali and alkaline earth metals, are normally considered basic and may conventionally be considered detrimental to catalyst performance. The presence of these species, however, surprisingly and unexpectedly, may be beneficial to the catalyst performance. In some embodiments, these species may act as catalyst promoters or a necessary part of the acidic catalyst structure such in layered or sheet silicates such as montmorillonite. Without being bound by theory, it is postulated that these cations create a strong dipole with species that create acidity.

Additional modifiers that may be included in the catalyst include, for example, boron, aluminum, magnesium, zirconium, and hafnium.

As will be appreciated by those of ordinary skill in the art, the support materials, if included in the catalyst of the present invention, preferably are selected such that the catalyst system is suitably active, selective and robust under the process conditions employed for the formation of the desired product, e.g., acrylic acid or alkyl acrylate. Also, the active metals and/or pyrophosphates that are included in the catalyst of the invention may be dispersed throughout the support, coated on the outer surface of the support (egg shell) or decorated on the surface of the support. In some embodiments, in the case of macro- and meso-porous materials, the active sites may be anchored or applied to the surfaces of the pores that are distributed throughout the particle and hence are surface sites available to the reactants but are distributed throughout the support particle.

The inventive catalyst may further comprise other additives, examples of which may include: molding assistants for enhancing moldability; reinforcements for enhancing the strength of the catalyst; pore-forming or pore modification agents for formation of appropriate pores in the catalyst, and binders. Examples of these other additives include stearic acid, graphite, starch, cellulose, silica, alumina, glass fibers, silicon carbide, and silicon nitride. Preferably, these additives do not have detrimental effects on the catalytic performances, e.g., conversion and/or activity. These various additives may be added in such an amount that the physical strength of the catalyst does not readily deteriorate to such an extent that it becomes impossible to use the catalyst practically as an industrial catalyst.

### Separation

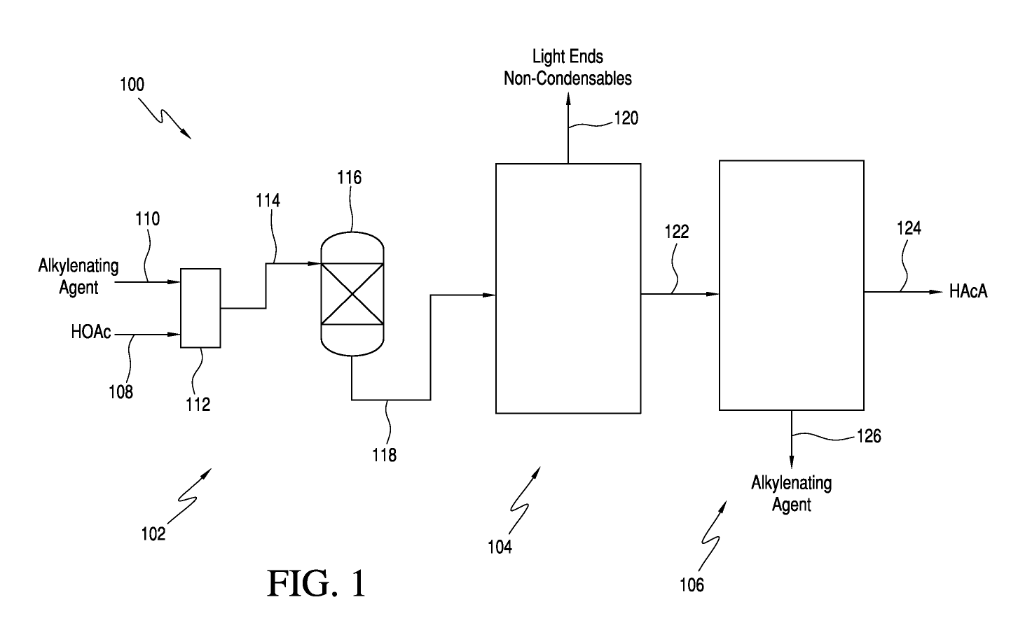

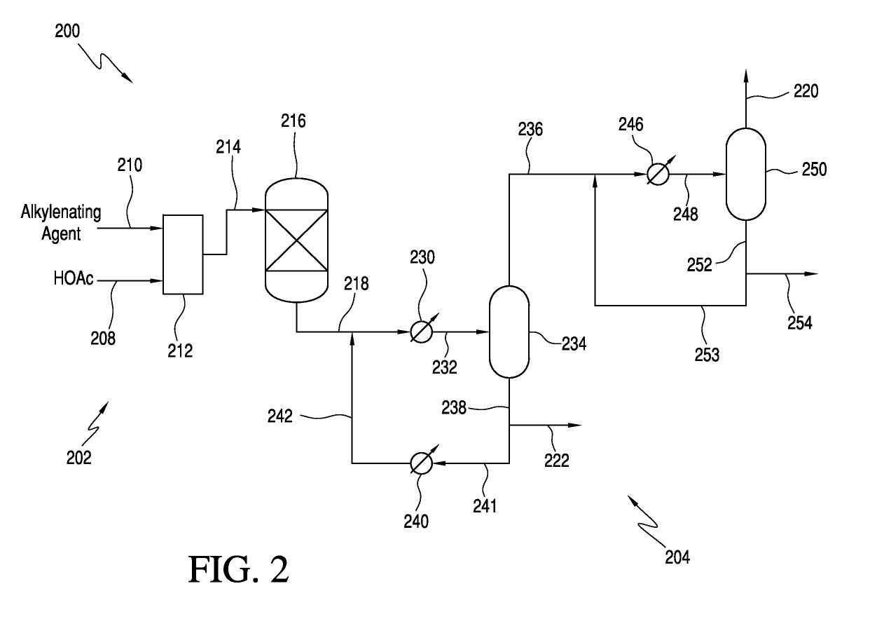

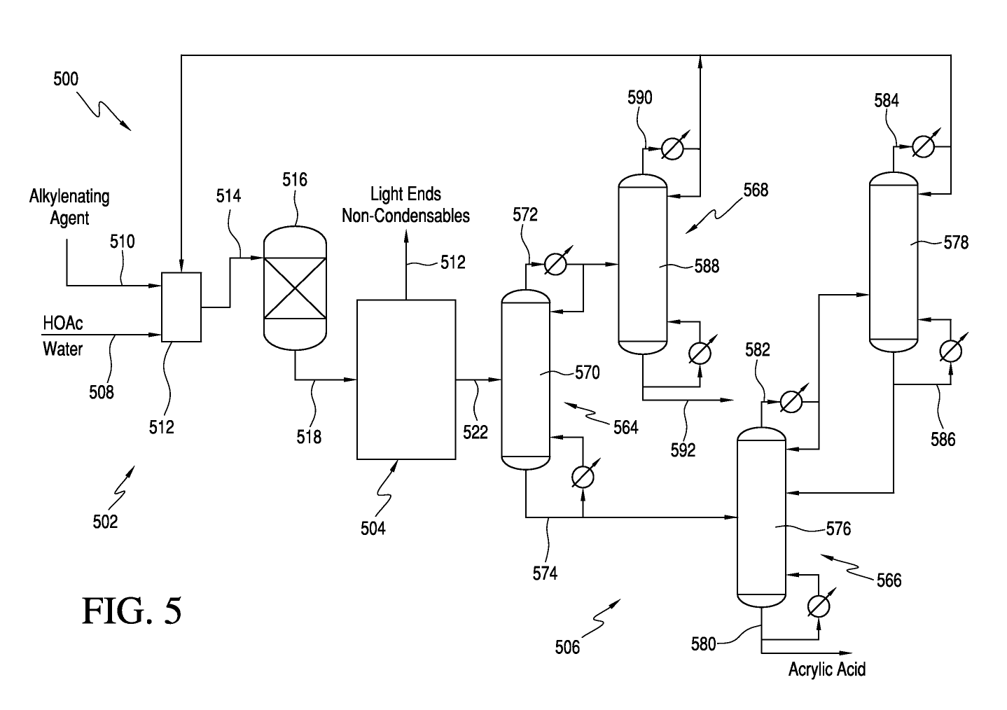

As discussed above, the crude product stream is separated to yield an intermediate acrylate product stream. FIG. 1 is a flow diagram depicting the formation of the crude product stream and the separation thereof to obtain an intermediate acrylate product stream. FIGS. 2-4 illustrate three different options for removing light ends and non-condensable gases from the crude acrylate product. FIG. 5 illustrates a separation scheme for separating acrylic acid and water from the crude acrylate product.

As shown in FIG. 1, acrylate product system 100 comprises reaction zone 102, light ends and non-condensable gases removal zone 104 and alkylenating agent split zone 106. Reaction zone 102 comprises reactor 116, alkanoic acid feed 108, e.g., acetic acid feed, alkylenating agent feed 110, e.g., formaldehyde feed, and vaporizer 112.

Acetic acid and formaldehyde are fed to vaporizer 112 via lines 108 and 110, respectively, to create a vapor feed stream, which exits vaporizer 112 via line 114 and is directed to reactor 116. In one embodiment, lines 108 and 110 may be combined and jointly fed to the vaporizer 112. The temperature of the vapor feed stream in line 114 is preferably from 200° C. to 600° C., e.g., from 250° C. to 500° C. or from 340° C. to 425° C. Alternatively, a vaporizer may not be employed and the reactants may be fed directly to reactor 106.

Any feed that is not vaporized may be removed from vaporizer 112 and may be recycled or discarded. In addition, although line 114 is shown as being directed to the upper half of reactor 116, line 114 may be directed to the middle or bottom of first reactor 106. Further modifications and additional components to reaction zone 102 and alkylenating agent split zone 106 are described below.

Reactor 116 contains the catalyst that is used in the reaction to form crude product stream, which is withdrawn, preferably continuously, from reactor 116 via line 116. Although FIG. 1 shows the crude product stream being withdrawn from the bottom of reactor 116, the crude product stream may be withdrawn from any portion of reactor 116. Exemplary composition ranges for the crude product stream are shown in Table 1 above.

In one embodiment, one or more guard beds (not shown) may be used upstream of the reactor to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens.

The crude product stream in line 118 is fed to light ends and non-condensable gases removal unit 104 to yield vapor stream 120 and a condensed crude product stream 122. Removal unit 104 may comprise heat exchanges and/or separation units, such as distillation columns and flashers, as shown n in FIGS. 2-4. In one example, removal until 104 comprises shell and tube heat exchanger. In one example, removal unit 104 comprises a rectifying column. In one example, removal unit 104 comprises a quench column. In an embodiment, polymerization inhibitor may be added during light ends and non-condensable gases removal to prevent the polymerization of acrylic products in the condensed product stream. Vapor stream 120 is removed from the acrylate production system and optionally flared or purged.

The condensed crude product stream in line 122 is fed to alkylenating agent split unit 106. Alkylenating agent split unit 106 may comprise one or more separation units, e.g., two or more or three or more. In one example, the alkylenating agent split unit contains multiple columns, as shown in FIG. 5. Alkylenating agent split unit 106 separates the crude product stream into at least one intermediate acrylate product stream, which exits via line 124 and at least one alkylenating agent stream, which exits via line 126. Exemplary compositional ranges for the intermediate acrylate product stream are shown in Table 2. Components other than those listed in Table 2 may also be present in the intermediate acrylate product stream.

TABLE 2
INTERMEDIATE ACRYLATE PRODUCT STREAM COMPOSITION

| | Conc. (wt. %) | Conc. (wt. %) | Conc. (wt. %) |
|---|---|---|---|
| Acrylic Acid | at least 5 | 5 to 99 | 35 to 65 |
| Acetic Acid | less than 95 | 5 to 90 | 20 to 60 |
| Water | less than 25 | 0.1 to 10 | 0.5 to 7 |
| Alkylenating Agent | <1 | <0.5 | <0.1 |
| Propionic Acid | <10 | 0.01 to 5 | 0.01 to 1 |

In other embodiments, the intermediate acrylate product stream comprises higher amounts of alkylenating agent. For example, the intermediate acrylate product stream may comprise from 1 wt. % to 10 wt. % alkylenating agent, e.g., from 1 wt. % to 8 wt. % or from 2 wt. % to 5 wt. %. In one embodiment, the intermediate acrylate product stream comprises greater than 1 wt. % alkylenating agent, e.g., greater than 5 wt. % or greater than 10 wt. %.

Exemplary compositional ranges for the alkylenating agent stream are shown in Table 3. Components other than those listed in Table 3 may also be present in the purified alkylate product stream.

TABLE 3
ALKYLENATING AGENT STREAM COMPOSITION

| | Conc. (wt. %) | Conc. (wt. %) | Conc. (wt. %) |
|---|---|---|---|
| Acrylic Acid | less than 15 | 0.01 to 10 | 0.1 to 5 |
| Acetic Acid | 10 to 65 | 20 to 65 | 25 to 55 |
| Water | 15 to 75 | 25 to 65 | 30 to 60 |
| Alkylenating Agent | at least 1 | 1 to 75 | 10 to 20 |
| Propionic Acid | <10 | 0.001 to 5 | 0.001 to 1 |

In other embodiments, the alkylenating stream comprises lower amounts of acetic acid. For example, the alkylenating agent stream may comprise less than 10 wt. % acetic acid, e.g., less than 5 wt. % or less than 1 wt. %.

FIG. 2 illustrates the separation of light ends and non-condensables from the crude acrylate product stream in accordance with the present invention. In an embodiment, the crude acrylate product stream is cooled in one or more stages using heat exchangers. In another embodiment, one or more cooled streams, e.g., derivatives of the crude acrylate product stream, may be returned and combined with the crude acrylate product stream. The resultant cooling of the crude acrylate product stream may prevent acrylic acid polymerization. In another embodiment, polymerization inhibitors may be used to prevent acrylic acid polymerization.

As shown in FIG. 2, acrylate product system 200 comprises reaction zone 202 and light ends and non-condensable removal zone 204. Reaction zone 202 comprises reactor 216, alkanoic acid feed 208, e.g., acetic acid feed, alkylenating agent feed 210, e.g., formaldehyde feed, vaporizer 212, and line 214. Reaction zone 202 and the components thereof function in a manner similar to reaction zone 102 in FIG. 1. Removal zone 204 comprises one or more heat exchangers and one or more flashers.

Reaction zone 202 yields a crude product stream, which exits reaction zone 202 via line 218 and is directed to removal zone 204. The components of the crude product stream are discussed above. Removal zone 204 separates light ends and non-condensable gases from the crude product stream to yield condensed crude product streams in lines 222 and 254 and light ends and non-condensable gases in cooled vapor stream 220. Condensed crude product streams in lines 222 and 254 may be combined and fed to an alkylenating split unit, as shown in FIG. 5.

Crude product stream 218 exits reactor 216 and is feed to a separation unit comprising heat exchanger 230 and flasher 234. Crude product stream 218 is cooled in heat exchanger 230 to yield first cooled stream 232. First cooled stream 232 has a lower temperature than crude product stream 218. First cooled stream 232 is fed to first flasher 234 where stream 232 is separated into first vapor stream 236 and first liquid stream 238. First vapor stream 236 comprises light ends, non-condensable gases, and acrylate products. In an embodiment, first liquid stream 238 comprises a majority of the condensable components of the crude acrylate product stream, e.g., more than 55 wt. %, more than 70 wt. %, or more than 85 wt. %.

A portion of first liquid stream 238 may be used to cool the crude acrylate product. In an embodiment, first liquid stream 238 is split into first liquid pump-around stream 241 and condensed acrylate product stream 222. In an embodiment, first pump-around stream 241 may be fed to second heat exchanger 240 to yield cooled first pump-around stream 242. Cooled first pump-around stream 242 may be combined with crude product stream 218 to reduce the temperature of crude product stream 218 and fed to first heat exchanger 230. As a result of mixing the cooled first pump-around stream 242 with the crude acrylate product, it requires less heat exchange area to accomplish the cooling required and thereby reduces the size of the heat exchanger. In one embodiment, the cooled first pump-around stream 242 and crude product stream 218 are introduced separately to first heat exchanger 230. In another embodiment, the temperature of first pump-around stream 242 is lower than crude product stream 218. First pump-around stream 242 may be combined with crude product stream and fed to flasher 234 without passing through heat exchanger 230.

In addition to reducing the temperature of the crude acrylate product stream, the use of cooled first pump-around stream also prevents acrylic acid in the crude acrylate product from undergoing polymerization in the heat exchanger. In an embodiment, one or more polymerization inhibitor may be added to first liquid pump-around stream 242 to prevent acrylic acid polymerization. Examples of useful polymerization inhibitors are described above.

Returning to first flasher 234, first vapor stream 236 exits first flasher 234 and is fed to a second separation unit comprising third heat changer 246 and second flasher 250. First vapor stream 236 enters third heat exchanger 246 to yield second cooled stream 248. Second cooled stream 248 has a lower temperature than first vapor stream 236. Second cooled stream 248 is fed to second flasher 250 where it is separated into cooled vapor stream 220 and second liquid stream 252. Cooled vapor stream 220 comprises light ends and non-condensable gases, as discussed above, and may be removed from acrylate product system 200 and/or may be incinerated.

A portion of second liquid stream 252 may be returned and combined with first vapor stream 236. In one embodiment, second liquid stream 252 is separated into a second liquid pump-around stream 253 and condensed crude product stream 254. Second liquid pump-around stream 253 has a lower temperature than first vapor stream 236 and may be returned and combined with first vapor stream 236. The combining of the two streams reduced the temperature of the first vapor stream 236 and reduces the heat exchange area required to accomplish the cooling requirement. In another embodiment, a heat exchanger maybe used to cool the second liquid pump-around stream 253 before it is combined with first vapor stream 236.

Condensed product streams 222 and 254 from first and second flashers 234 and 250 may be fed to alkylenating agent split zone, as discussed below in connection with FIG. 5.

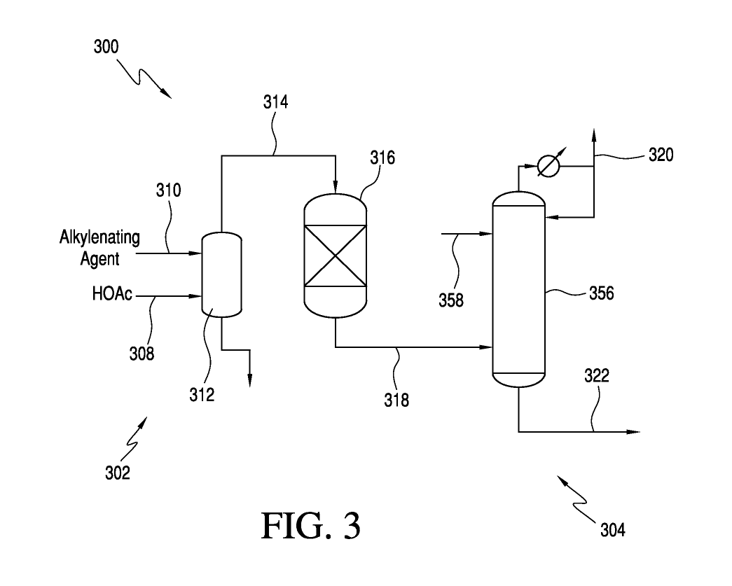

FIG. 3 illustrates the removal of light ends and non-condensable gases from the crude acrylate product using a rectifying column. As shown in FIG. 3, acrylate product system 300 comprises reaction zone 302 and light ends and non-condensable removal zone 304. Reaction zone 302 comprises reactor 316, acetic acid feed 308, formaldehyde feed 310, vaporizer 312, and line 314. Reaction zone 302 and the components thereof function in a manner similar to reaction zone 102 in FIG. 1. Removal zone 304 comprises one or more separation columns, e.g., a rectifying column.

Reaction zone 302 yields a crude product stream, which exits reaction zone 302 via line 318 and is directed to removal zone 304. The components of the crude product stream are discussed above. Removal zone 304 separates the crude product stream to yield vapor stream 320 and residue stream 322. Residue stream 322 may be considered a condensed product stream. Vapor stream 320 comprises light ends and non-condensable gases and may be removed from acrylate product system 300. Portions of vapor stream 320 may be incinerated or recycled back to the reactor.

As shown in FIG. 3, crude acrylate product 318 is introduced to column 356, preferably in the lower part of column 356, e.g., lower third, or lower quarter. Preferably, column 356 is a rectifying distillation column. In one embodiment, polymerization inhibitor may be added to column 356 via line 358. Polymerization inhibitors may be used to prevent the polymerization of acrylic acid in the crude acrylate product. Examples of polymerization inhibitor are discussed above.

Column 356 may be a tray or packed column. In one embodiment, column 356 is a tray column having from 5 to 70 trays, e.g., from 15 to 50 trays or from 20 to 45 trays. Although the temperature and pressure of column 356 may vary, when at atmospheric pressure the temperature of the residue exiting in line 322 preferably is from 50° C. to 150° C., e.g., from 75° C. to 130° C. or from 90° C. to 115° C. The temperature of the vapor exiting in line 320 from column 356 preferably is from 0° C. to 70° C., e.g., from 20° C. to 60° C. or from 30° C. to 50° C. Column 356 may operate at atmospheric pressure. In other embodiments, the pressure of column 356 may range from 10 kPa to 110 kPa, e.g., from 50 kPa to 110 kPa or from 90 kPa to 110 kPa.

In an embodiment, the distillate of column 356 preferably is refluxed as shown in FIG. 3, for example, at a reflux ratio from 1:10 to 10:1, e.g., from 1:3 to 3:1 or from 1:2 to 2:1. In an embodiment, no reboiler is used with column 356. As such, the potential for acrylic polymerization is reduced. Residue 322 exits column 356 and is introduced to alkylenating agent split zone, as discussed below in FIG. 5.

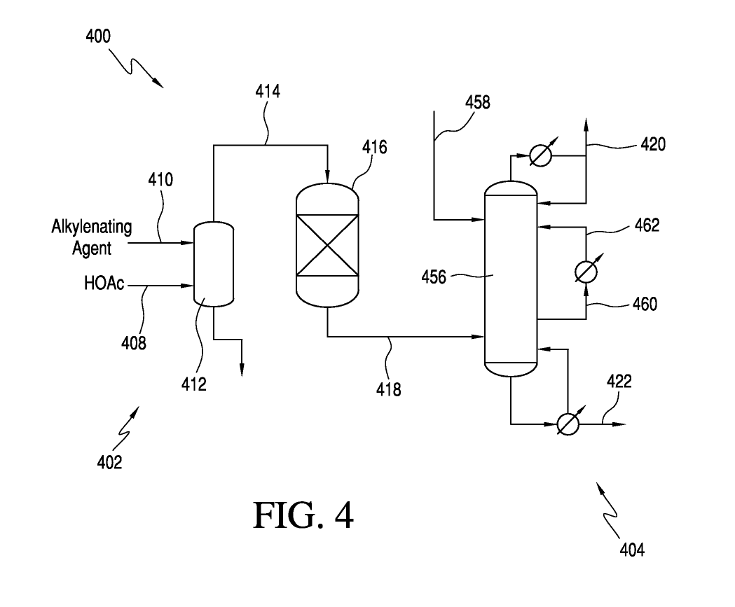

In another embodiment, column 356 may be a quench column. FIG. 4 illustrates the removal of light ends and non-condensable gases from the crude acrylate product using a quench column in line 420. As shown in FIG. 4, crude acrylate product 418 is introduced to quench column 456, preferably in the lower part of column 456, e.g., lower third, or lower quarter. Quench column 456 separates crude product stream 418 to yield vapor stream 420 and residue stream 422. A quenching agent may be added to column 456 via line 458, preferably in the upper part of column 456, e.g., upper third, or upper quarter. The quenching agent may be a solvent, such as water, acetic acid, or other suitable solvent. Preferably, the solvent is at a temperature lower than crude acrylate produce stream 418. In an embodiment, the solvent is at ambient temperature.

In an embodiment, one or more pump-around stream may be used to aid with the cooling of crude acrylate product 418. For example, side stream 460 may be withdrawn from column 456 and fed through a heat exchanger to yield a cooled side stream 462. Cooled side stream 462 is returned to the column at a position below quenching agent 458.

Column 456 may be a tray or packed column. In one embodiment, column 456 is a tray column having from 5 to 70 trays, e.g., from 15 to 50 trays or from 20 to 45 trays. Although the temperature and pressure of column 456 may vary, when at atmospheric pressure the temperature of the residue exiting in line 422 preferably is from 50° C. to 150° C., e.g., from 75° C. to 130° C. or from 90° C. to 115° C. The temperature of the vapor exiting in line 420 from column 456 preferably is from 0° C. to 70° C., e.g., from 20° C. to 60° C. or from 30° C. to 50° C. Column 456 may operate at atmospheric pressure. In other embodiments, the pressure of column 456 may range from 10 kPa to 110 kPa, e.g., from 50 kPa to 110 kPa or from 90 kPa to 110 kPa.

In an embodiment, the distillate of column 456 preferably is refluxed as shown in FIG. 4, for example at a reflux ratio from 1:10 to 10:1, e.g., from 1:3 to 3:1 or from 1:2 to 2:1. In an embodiment, the residue of column 456 preferably is reboiled as shown in FIG. 4, for example at a reboil ratio from 1:10 to 10:1, e.g., from 1:3 to 3:1 or from 1:2 to 2:1. In an embodiment, one or more polymerization inhibitor may be added to column 456 to prevent the polymerization of acrylic acid. Residue 422 exits column 456 and is introduced to alkylenating agent split zone, as discussed below.

FIG. 5 shows an overview of one reaction/separation scheme in accordance with the present invention. The separation zone of FIG. 5 is merely exemplary and other suitable separation zones may be utilized. Acrylate product system 500 comprises reaction zone 502 and separation zone 504. Reaction zone 502 comprises reactor 506, acetic acid feed 508, formaldehyde feed 510, vaporizer 512, and line 514. Reaction zone 502 and the components thereof function in a manner similar to reaction zones of FIGS. 1-4.

Reaction zone 502 yields a crude product stream, which exits reaction zone 502 via line 518 and is directed to separation zone 504. The components of the crude product stream are discussed above. Separation zone 504 comprises light ends and non-condensable gases removal zone 504, alkylenating agent split unit 564, acrylate product split unit 566, and drying unit 568. In accordance with an embodiment of the present invention, crude acrylate product in line 518 is introduced to light ends and non-condensable gases removal zone 504 to remove light ends and condensable gases and to yield a condensed crude product stream in line 522 as discussed above. The condensed crude stream in line 522 comprises the acrylic acid, acetic acid, alkylenating agent, and/or water, which are introduced to alkylenating agent split unit 564.

Alkylenating agent split unit 564 may comprise any suitable separation device or combination of separation devices. For example, alkylenating agent split unit 564 may comprise a column, e.g., a standard distillation column, an extractive distillation column and/or an azeotropic distillation column. In other embodiments, alkylenating agent split unit 564 comprises a precipitation unit, e.g., a crystallizer and/or a chiller. Preferably, alkylenating agent split unit 564 comprises a single distillation column.

In another embodiment, the alkylenating agent split is performed by contacting the crude product stream with a solvent that is immiscible with water. For example, alkylenating agent split unit 564 may comprise at least one liquid-liquid extraction column. In another embodiment, the alkylenating agent split is performed via azeotropic distillation, which employs an azeotropic agent. In these cases, the azeotropic agent may be selected from the group consisting of methyl isobutylketene, o-xylene, toluene, benzene, n-hexane, cyclohexane, p-xylene, and mixtures thereof. This listing is not exclusive and is not meant to limit the scope of the invention. In another embodiment, the alkylenating agent split is performed via a combination of distillations, e.g., standard distillation, and crystallization. Of course, other suitable separation devices may be employed either alone or in combination with the devices mentioned herein.

In FIG. 5, alkylenating agent split unit 564 comprises first column 570. The condensed crude liquid stream in line 522 is directed to first column 570. First column 570 separates the condensed crude product stream to form a distillate in line 572 and a residue in line 574. The distillate may be refluxed and the residue may be boiled up as shown. Distillate stream 572 comprises at least 1 wt % alkylenating agent. As such, distillate stream 572 may be considered an alkylenating agent stream. The residue exits first column 570 in line 574 and comprises a significant portion of acrylate product. As such, residue stream 574 is an intermediate product stream. In one embodiment, at least a portion of distillate stream 572 is directed to drying unit 568.

Exemplary compositional ranges for the distillate and residue of first column 570 are shown in Table 4. Components other than those listed in Table 4 may also be present in the residue and distillate.

TABLE 4
FIRST COLUMN

| | Conc. (wt. %) | Conc. (wt. %) | Conc. (wt. %) |
|---|---|---|---|
| Distillate | | | |
| Acrylic Acid | <5 | <3 | 0.01 to 3 |
| Acetic Acid | <10 | <5 | 0.01 to 5 |
| Water | >50 | 50 to 90 | 60 to 85 |
| Alkylenating Agent | >5 | 5 to 50 | 10 to 30 |
| Propionic Acid | <1 | <0.1 | <0.01 |
| Residue | | | |
| Acrylic Acid | 5 to 75 | 10 to 60 | 20 to 45 |
| Acetic Acid | 20 to 80 | 30 to 70 | 40 to 60 |
| Water | <10 | 0.01 to 10 | 0.1 to 5 |
| Alkylenating Agent | <30 | 0.01 to 30 | 1 to 15 |
| Propionic Acid | <1 | <0.1 | <0.01 |

In one embodiment, the first distillate comprises smaller amounts of acetic acid, e.g., less than 25 wt. %, less than 10 wt. %, less than 5 wt. % or less than 1 wt. %. In one embodiment, the first residue comprises larger amounts of alkylenating agent.

In some embodiments, the intermediate acrylate product stream comprises higher amounts of alkylenating agent, e.g., greater than 1 wt. % greater than 5 wt. % or greater than 10 wt. %.

For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

In one embodiment, polymerization inhibitors and/or anti-foam agents may be employed in the separation zone, e.g., in the units of the separation zone. The inhibitors may be used to reduce the potential for fouling caused by polymerization of acrylates. The anti-foam agents may be used to reduce potential for foaming in the various streams of the separation zone. The polymerization inhibitors and/or the anti-foam agents may be used at one or more locations in the separation zone.

In cases where any of alkylenating agent split unit 564 comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 300 kPa, e.g., from 10 kPa to 100 kPa or from 40 kPa to 80 kPa. In preferred embodiments, the pressure at which the column(s) are operated is kept at a low level e.g., less than 100 kPa, less than 80 kPa, or less than 60 kPa. In terms of lower limits, the column(s) may be operated at a pressures of at least 1 kPa, e.g., at least 20 kPa or at least 40 kPa. Without being bound by theory, it is believed that alkylenating agents, e.g., formaldehyde, may not be sufficiently volatile at lower pressures. Thus, maintenance of the column pressures at these levels surprisingly and unexpectedly provides for efficient separation operations. In addition, it has surprisingly and unexpectedly been found that by maintaining a low pressure in the columns of alkylenating agent split unit 564 may inhibit and/or eliminate polymerization of the acrylate products, e.g., acrylic acid, which may contribute to fouling of the column(s).

In one embodiment, the alkylenating agent split is achieved via one or more liquid-liquid extraction units. Preferably, the one or more liquid-liquid extraction units employ one or more extraction agents. Multiple liquid-liquid extraction units may be employed to achieve the alkylenating agent split. Any suitable liquid-liquid extraction devices used for multiple equilibrium stage separations may be used. Also, other separation devices, e.g., traditional columns, may be employed in conjunction with the liquid-liquid extraction unit(s).

In one embodiment (not shown), the crude product stream is fed to a liquid-liquid extraction column where the crude product stream is contacted with an extraction agent, e.g., an organic solvent. The liquid-liquid extraction column extracts the acids, e.g., acrylic acid and acetic acid, from the crude product stream. An aqueous phase comprising water, alkylenating agent, and some acetic acid exits the liquid-liquid extraction unit. Small amounts of acrylic acid may also be present in the aqueous stream. The aqueous phase may be further treated and/or recycled. An organic phase comprising acrylic acid, acetic acid, and the extraction agent also exits the liquid-liquid extraction unit. The organic phase may also comprise water and formaldehyde. The acrylic acid may be separated from the organic phase and collected as product. The acetic acid may be separated then recycled and/or used elsewhere. The solvent may be recovered and recycled to the liquid-liquid extraction unit.

The inventive process further comprises the step of separating the intermediate acrylate product stream to form a finished acrylate product stream and a first finished acetic acid stream. The finished acrylate product stream comprises acrylate product(s) and the first finished acetic acid stream comprises acetic acid. The separation of the acrylate products from the intermediate product stream to form the finished acrylate product may be referred to as the “acrylate product split.”

Returning to FIG. 5, intermediate product stream 574 exits alkylenating agent split unit 564 and is directed to acrylate product split unit 566 for further separation, e.g., to further separate the acrylate products therefrom. Acrylate product split unit 566 may comprise any suitable separation device or combination of separation devices. For example, acrylate product split unit 566 may comprise at least one column, e.g., a standard distillation column, an extractive distillation column and/or an azeotropic distillation column. In other embodiments, acrylate product split unit 566 comprises a precipitation unit, e.g., a crystallizer and/or a chiller. Preferably, acrylate product split unit 566 comprises two standard distillation columns as shown in FIG. 5. In another embodiment, acrylate product split unit 566 comprises a liquid-liquid extraction unit. Of course, other suitable separation devices may be employed either alone or in combination with the devices mentioned herein.

In FIG. 5, acrylate product split unit 566 comprises second column 576 and third column 578. Acrylate product split unit 566 receives at least a portion of intermediate product stream in line 574 and separates same into finished acrylate product stream 580 and at least one acetic acid-containing stream. As such, acrylate product split unit 566 may yield the finished acrylate product.

As shown in FIG. 5, at least a portion of purified acrylic product stream in line 580 is directed to second column 576. Second column 576 separates the purified acrylic product stream to form second distillate, e.g., line 582, and second residue, which is the finished acrylate product stream, e.g., line 580. The distillate may be refluxed and the residue may be boiled up as shown.

Stream 582 comprises acetic acid and some acrylic acid. The second column residue exits second column 576 in line 580 and comprises a significant portion of acrylate product. As such, stream 580 is a finished product stream. Exemplary compositional ranges for the distillate and residue of second column 576 are shown in Table 5. Components other than those listed in Table 5 may also be present in the residue and distillate.

TABLE 5
SECOND COLUMN

| | Conc. (wt. %) | Conc. (wt. %) | Conc. (wt. %) |
|---|---|---|---|
| Distillate | | | |
| Acrylic Acid | 0.1 to 50 | 1 to 30 | 5 to 30 |
| Acetic Acid | 60 to 95 | 70 to 90 | 75 to 85 |
| Water | 0.01 to 15 | 0.1 to 10 | 1 to 5 |
| Alkylenating Agent | 0.01 to 25 | 0.01 to 15 | 0.1 to 10 |
| Propionic Acid | <1 | <0.1 | <0.01 |
| Residue | | | |
| Acrylic Acid | 75 to 99.99 | 85 to 99.9 | 95 to 99.5 |
| Acetic Acid | 0.01 to 15 | 0.1 to 10 | 0.1 to 5 |
| Water | <1 | <0.1 | <0.01 |
| Alkylenating Agent | <1 | 0.001 to 1 | 0.1 to 1 |
| Propionic Acid | <1 | <0.1 | <0.01 |

Returning to FIG. 5, at least a portion of stream 582 is directed to third column 576. Third column 576 separates the at least a portion of stream 574 into a distillate in line 582 and a residue in line 580. The distillate may be refluxed and the residue may be boiled up as shown. The distillate comprises a major portion of acetic acid. In one embodiment, at least a portion of line 584 is returned, either directly or indirectly, to reactor 516. The third column residue exits third column 578 in line 586 and comprises acetic acid and some acrylic acid. At least a portion of line 586 may be returned to second column 576 for further separation. In one embodiment, at least a portion of line 586 is returned, either directly or indirectly, to reactor 516. In another embodiment, at least a portion of the acetic acid-containing stream in either or both of lines 584 and 586 may be directed to an ethanol production system that utilizes the hydrogenation of acetic acid to form the ethanol. In another embodiment, at least a portion of the acetic acid-containing stream in either or both of lines 584 and 586 may be directed to a vinyl acetate system that utilizes the reaction of ethylene, acetic acid, and oxygen form the vinyl acetate. Exemplary compositional ranges for the distillate and residue of third column 578 are shown in Table 6. Components other than those listed in Table 6 may also be present in the residue and distillate.

TABLE 6
THIRD COLUMN

| | Conc. (wt. %) | Conc. (wt. %) | Conc. (wt. %) |
|---|---|---|---|
| Distillate | | | |
| Acrylic Acid | 0.01 to 10 | 0.05 to 5 | 0.1 to 1 |
| Acetic Acid | 60 to 99.9 | 70 to 99.5 | 80 to 99 |
| Water | 0.01 to 20 | 0.1 to 15 | 1 to 10 |
| Alkylenating Agent | 0.001 to 40 | 0.01 to 25 | 0.1 to 15 |
| Propionic Acid | <1 | <0.1 | <0.01 |
| Residue | | | |
| Acrylic Acid | 5 to 50 | 15 to 40 | 20 to 35 |
| Acetic Acid | 50 to 90 | 60 to 80 | 65 to 75 |
| Water | 0.001 to 10 | 0.01 to 5 | 0.1 to 1 |
| Alkylenating Agent | 0.001 to 5 | 0.001 to 1 | 0.05 to 1 |
| Propionic Acid | <1 | <0.1 | <0.01 |

In cases where the acrylate product split unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 300 kPa, e.g., from 10 kPa to 100 kPa or from 40 kPa to 80 kPa. In preferred embodiments, the pressure at which the column(s) are operated is kept at a low level e.g., less than 50 kPa, less than 27 kPa, or less than 20 kPa. In terms of lower limits, the column(s) may be operated at a pressures of at least 1 kPa, e.g., at least 3 kPa or at least 5 kPa. Without being bound by theory, it has surprisingly and unexpectedly been found that be maintaining a low pressure in the columns of acrylate product split unit 580 may inhibit and/or eliminate polymerization of the acrylate products, e.g., acrylic acid, which may contribute to fouling of the column(s).

It has also been found that, surprisingly and unexpectedly, maintaining the temperature of acrylic acid-containing streams fed to acrylate product split unit 580 at temperatures below 140° C., e.g., below 130° C. or below 115° C., may inhibit and/or eliminate polymerization of acrylate products. In one embodiment, to maintain the liquid temperature at these temperatures, the pressure of the column(s) is maintained at or below the pressures mentioned above. In these cases, due to the lower pressures, the number of theoretical column trays is kept at a low level, e.g., less than 10, less than 8, less than 7, or less than 5. As such, it has surprisingly and unexpectedly been found that multiple columns having fewer trays inhibit and/or eliminate acrylate product polymerization. In contrast, a column having a higher amount of trays, e.g., more than 10 trays or more than 15 trays, would suffer from fouling due to the polymerization of the acrylate products. Thus, in a preferred embodiment, the acrylic acid split is performed in at least two, e.g., at least three, columns, each of which have less than 10 trays, e.g. less than 7 trays. These columns each may operate at the lower pressures discussed above.

Returning to FIG. 5, alkylenating agent stream 572 exits alkylenating agent split unit 564 and is directed to drying unit 568 for further separation, e.g., to further separate the water therefrom. The separation of the formaldehyde from the water may be referred to as dehydration. Drying unit 568 may comprise any suitable separation device or combination of separation devices. For example, drying unit 568 may comprise at least one column, e.g., a standard distillation column, an extractive distillation column and/or an azeotropic distillation column. In other embodiments, drying unit 568 comprises a dryer and/or a molecular sieve unit. In a preferred embodiment, drying unit 568 comprises a liquid-liquid extraction unit. In one embodiment, drying unit 568 comprises a standard distillation column as shown in FIG. 5. Of course, other suitable separation devices may be employed either alone or in combination with the devices mentioned herein.

In FIG. 5, drying unit 568 comprises fourth column 588. Drying unit 568 receives at least a portion of alkylenating agent stream in line 572 and separates same into a fourth distillate comprising water and formaldehyde in line 590 and a fourth residue comprising mostly water in line 592. The distillate may be refluxed and the residue may be boiled up as shown. In one embodiment, at least a portion of line 590 is returned, either directly or indirectly, to reactor 516.

In one embodiment, depending on the amount of methanol in alkylenating agent stream 572, the acrylate product system 500 may include a methanol removal unit (not shown) for further separation, e.g., to further separate the methanol therefrom. Methanol removal unit may comprise any suitable separation device or combination of separation devices. For example, methanol removal unit may comprise at least one column, e.g., a standard distillation column, an extractive distillation column and/or an azeotropic distillation column. In one embodiment, methanol removal unit comprises a liquid-liquid extraction unit. In a preferred embodiment, methanol removal unit comprises a standard distillation column. Of course, other suitable separation devices may be employed either alone or in combination with the devices mentioned herein. Methanol removal unit receives at least a portion of alkylenating agent and separates same into a distillate comprising methanol and water and a residue comprising water and formaldehyde. The distillate may be refluxed and the residue may be boiled up (not shown). In one embodiment, at least a portion of the formaldehyde in the residue is returned, either directly or indirectly, to reaction system. The distillate may be used to form additional formaldehyde.

Exemplary compositional ranges for the distillate and residue of fourth column 544 are shown in Table 7. Components other than those listed in Table 7 may also be present in the residue and distillate.

TABLE 7
FOURTH COLUMN

| | Conc. (wt. %) | Conc. (wt. %) | Conc. (wt. %) |
|---|---|---|---|
| Distillate | | | |
| Acrylic Acid | <1 | 0.001 to 5 | 0.01 to 1 |
| Acetic Acid | <1 | 0.001 to 5 | 0.01 to 1 |
| Water | 25 to 85 | 35 to 75 | 45 to 65 |
| Alkylenating Agent | 10 to 70 | 20 to 60 | 30 to 50 |
| Residue | | | |
| Acrylic Acid | <1 | 0.01 to 5 | 0.01 to 1 |
| Acetic Acid | 0.001 to 20 | 0.01 to 10 | 0.1 to 5 |
| Water | >60 | >70 | 80 to 99 |
| Alkylenating Agent | 0.0001 to 15 | 0.001 to 10 | 0.01 to 5 |
| Propionic Acid | <1 | <0.1 | <0.01 |

In cases where the drying unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 500 kPa, e.g., from 25 kPa to 400 kPa or from 100 kPa to 300 kPa.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing an acrylate product, the process comprising the steps of:
(a) providing a crude acrylate product stream comprising the acrylate product, an alkylenating agent, light ends, and non-condensable gases;
(b) separating in at least one separation unit the crude acrylate product stream without the addition of heat to form a cooled vapor stream comprising light ends and non condensable gases and at least one condensed crude product stream; and
(c) separating at least a portion of the at least one condensed crude product stream to form an alkylenating agent stream comprising at least 1 wt. % alkylenating agent and an intermediate product stream comprising acrylate product.

2. The process of claim 1, wherein step (b) comprises separating the crude acrylate product stream in a first separation unit to form a first vapor stream and a first liquid stream.

3. The process of claim 2, wherein the first separation unit comprises a heat exchanger and a flasher or knock-out pot.

4. The process of claim 2, wherein the first vapor stream comprises one or more light ends, non-condensable gases and condensable components.

5. The process of claim 2, wherein the first liquid stream comprises less than 1 wt. % light ends compounds.

6. The process of claim 2, further comprising:
adding inhibitors to the first liquid stream; and
cooling a portion of the first liquid stream to form a cooled first liquid pump around stream.

7. The process of claim 6, further comprising combining at least a portion of the cooled first liquid pump around stream with the crude acrylate product stream.

8. The process of claim 2, further comprising separating the first vapor stream in a second separation unit to form the cooled vapor stream and a second liquid stream.

9. The process of claim 8, wherein the cooled vapor stream contains less condensable components by weight percentage than the first vapor stream.

10. The process of claim 8, further comprising combining at least a portion of the second liquid stream with the first vapor stream to cool the first vapor stream.

11. The process of claim 1, wherein the cooled vapor stream comprises less than 5 wt. % acrylics.

12. The process of claim 9, wherein the second liquid stream comprises less than 1 wt. % light ends compounds.

13. The process of claim 1, wherein the at least one condensed crude product stream comprises less than 50 wt. % non-condensable gases.

14. The process of claim 1, wherein a temperature of the at least one condensed crude product stream is less than a temperature of the crude product stream.

15. The process of claim 1, wherein the at least one condensed crude product stream comprises at least 0.5 wt. % alkylenating agent.

16. The process of claim 1, wherein the at least one condensed crude product stream comprises less than 1 wt. % light ends.

17. A process of claim 1, wherein the separation unit is a rectifying column.

18. The process of claim 17, further comprising adding inhibitor to the rectifying column.

19. A process of claim 1, wherein the separation unit is a quench column.

20. The process of claim 19, further comprising feeding a solvent to the quench column and separating the crude acrylate product stream into a vapor stream and a residue stream.

21. The process of claim 20, wherein the residue stream comprises less than 1 wt. % light ends.

22. The process of claim 20, wherein the temperature of the solvent is lower than the temperature of the crude product stream.

23. The process of claim 19, further comprising adding inhibitor to the quench column.

24. The process of claim 19, wherein the quench column further comprises a pump around stream having an exit end at a lower portion of the quench column and a return end at a higher portion of the quench column.

25. The process of claim 24, wherein a temperature of the return end of pump around stream is lower than a temperature of the exit end of the pump around stream.

26. The process of claim 24, wherein a temperature of the return end of the pump around stream is reduced using a heat exchanger.
